(19) United States
(12) Patent Application Publication
(10) Pub. No.: US 2024/0151721 A1
(43) Pub. Date: May 9, 2024

(54) DETECTOR FOR DETECTING BACTERIA AND VIRUS, METHOD FOR DETECTING THE SAME AND LABEL KIT FOR THE SAME

(71) Applicant: UNIVERSITY PUBLIC CORPORATION OSAKA, Osaka (JP)

(72) Inventor: Hiroshi SHIIGI, Sakai-shi (JP)

(73) Assignee: UNIVERSITY PUBLIC CORPORATION OSAKA, Osaka (JP)

(21) Appl. No.: 18/548,564

(22) PCT Filed: Dec. 27, 2021
(86) PCT No.: PCT/JP2021/048534

§ 371 Date: Aug. 31, 2023

(30) Foreign Application Priority Data
Mar. 2, 2021 (JP) 2021-032552

(51) Int. Cl. G01N 33/569; G01N 33/543; G01N 33/553

(52) U.S. Cl. CPC G01N 33/56983; G01N 33/5438; G01N 33/553; G01N 33/56911

(57) ABSTRACT

A label corresponding to each of various bacteria and virus species is prepared, and a detector for detecting bacteria and/or virus in a sample to be tested is provided based on the attribute of the label. DPV detector includes an electrode chip, a voltage applying unit for applying a voltage in a predetermined range to the electrode chip, a current measuring unit for measuring a peak current value outputted from the electrode chip in accordance with the applied voltage, a data memory unit for storing in advance the peak current value and the applied voltage value at the time of the peak current for each of the plurality of types of metal nanostructures, a target specifying unit for comparing the measured data and the accumulated data of the current measuring unit to specify a target coupled to the metal nanostructure, and a displaying unit for displaying the specified target.

### TECHNICAL FIELD

The present invention relates to a detector, a method for detecting, a label kit, and a measurement kit for detecting bacteria, bacterial group, or virus.

### BACKGROUND OF THE INVENTION

Food poisoning caused by bacteria and virus is frequently occurring in our country, and stricter daily voluntary control by food business operators is necessary for the improvement of food safety.

Conventional detection techniques include, for example, colony counting and bioluminescence. Since the colony counting method uses agar and sheet medium, the operation is complicated and it takes one day to judge, and there is a problem in securing equipment and human resources. The bioluminescence method enables a rapid detection, but does not clarify the presence or absence of bacteria as an indicator of ATP shared by organisms, and does not satisfy the need to rapidly quantify bacteria on the spot.

In addition, the above-described detection technology does not correspond to a virus. In addition, in the prior art, such as immunochromatography and PCR methods (polymerase chain reaction), it is difficult to perform rapid testing, such as requiring culture and amplification, due to the fundamental limitations, and it is not satisfactory from the viewpoint of cost, such as labor cost and equipment cost.

The detection method of Patent Document 1 discloses a configuration of a nanoparticle-biomaterial complex, an extraction solution, a collector electrode, and a current peak measurement unit. More specifically, the nanoparticle-biomaterial complex includes one or more nanoparticles selected from the group of metals consisting of zinc, cadmium, lead, copper, gallium, arsenic, thallium, nickel, manganese, and bismuth, one or more bio-binding substances that are bound to the nanoparticles through a binding stabilizing substance and that specifically bind to the biomaterial to be detected, and a binding stabilizing substance that binds the nanoparticles to the bio-binding substances. The extraction solution separates and extracts the nanoparticles from the nanoparticle-biomaterial complex. The collection electrode collects nanoparticles from the extraction solution. The current peak measurement unit measures a corresponding current peak from the nanoparticles collected from the collection electrode. The biological material to be detected is a nucleic add such as DNA or RNA, an amino add, or a nucleic acid-amino add complex or an antibody.

Patent Document 2 discloses a manner for detecting a substance to be detected using a metal nanoparticle integrated structure. In the manner of Patent Document 2, a metal nanoparticle integrated structure and a metal nanostructure are introduced into a sample, the sample is irradiated with light, a spectrum of the sample is measured, and a detection target substance is detected based on the spectrum. The metal nanoparticle integrated structure includes a bead and a plurality of metal nanoparticles immobilized on a surface of the bead via an interaction site. The plurality of metal nanoparticles is modified with a first host molecule capable of specifically attaching a substance to be detected, and are spaced apart from each other by a distance equal to or smaller than the diameter of the metal nanoparticles. The metal nanostructures are modified with a second host molecule capable of specifically attaching the substance to be detected. The metal nanostructures are metal nanorods. The substance to be detected is an antigen. The first and second host molecules are antibodies that undergo an antigen-antibody reaction with an antigen. A “first host molecule” and a “second host molecule” are host molecules that can specifically attach to different sites of a substance to be detected. The material used for the beads is, for example, a resin such as acrylic, polyolefin, polyethylene, polypropylene, or polystyrene.

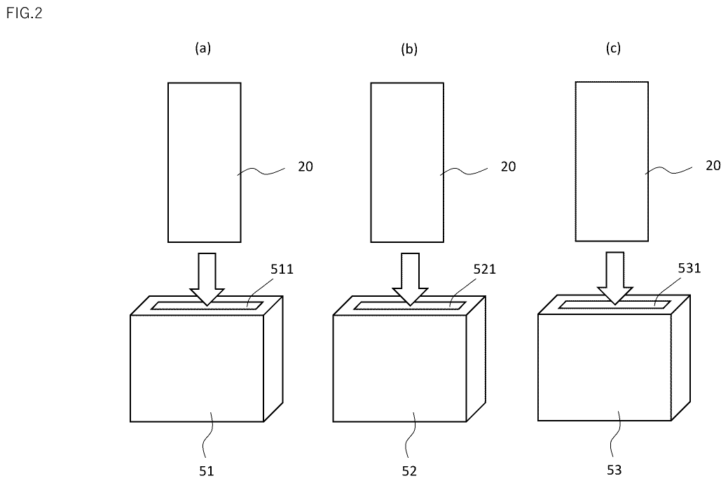

Patent Document 3 discloses a manner for detecting different bacterial pathogens. A gold nanoparticle is introduced with an antibody and a redox-active organic molecule to be used as a label, and the gold nanoparticle is specifically bound to an antigen on a bacterial surface by the antibody. The electrochemical detection system 107 shown in FIG. 2 is a detection mechanism that reads the redox current of organic molecules. In the examples of the specification, it is described to detect SA and PA using antibody-immobilized multi-array electrodes.

Patent Literature 4 is a system in which an electrochemical detection function is added to immunochromatography. Antibodies and electrochemically active species (EAC) are introduced into gold nanoparticles and used as labels. This system detects the current response of thionin, an electrochemically active species (EAC), when a label is bound to a target material through an antigen-antibody reaction.

Patent Document 5 discloses a biosensor capable of detecting whether a first target substance or a second target substance is contained in a sample according to the redox activity of a magnetic particle complex based on a change in voltage applied between the first electrode and the second electrode.

### PRIOR ART DOCUMENTS

[Patent Document]
[Patent Document 1] JP 2008-547016 JP
[Patent Document 2] Patent JP 5822239 B2
[Patent Document 3] U.S. Patent Publication U.S 20170199188 A
[Patent Document 4] International Patent Publication WO 2014/171891 A
[Patent Document 5] U.S. Pat. No. 11,041,854 B

### SUMMARY

### Summary of the Invention

### Problem to be Solved by the Invention

However, since the conventional detection method requires culturing, not only cost aspects such as labor cost and equipment cost, but also detection result is proven only after shipment, development of a simple and quick detection method is a problem in order to provide the food safely and inexpensively to the consumer. Usually, the sample liquid contains a plurality of types of bacteria and viruses, which are individually detected after a process including selective culturing and separation and purification. These steps have become a barrier in the speedup of detection.

Further, in Patent Document 1, although it is possible to perform multi-item simultaneous detection by changing the type of the nanoparticles, the aggregate of the nanoparticles is not used, and the detection target is limited to nucleic adds, amino acids, and the like.

Patent Document 2 uses metal nanoparticles for detection, but does not have a configuration in which multiple analytes or multiple items are simultaneously detected.

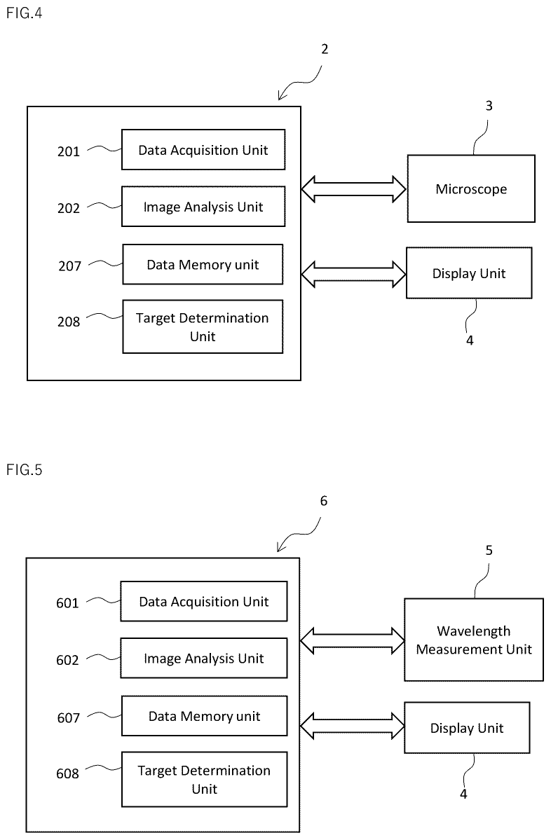

FIGS. 4A and 4B in Patent Document 3 shows the current responses for (A) S. aureus and (B) P. aeruginosa, respectively. Both are for (I) labeled bacteria, (II) bacteria only, and (III) labeling only. FIG. 5 is data of AMT labeled Pseudomonas aeruginosa (II) plasma in (I) aqueous solution, ATP labeled S. aureus in (III) aqueous solution, and ATP labeled S. aureus in (IV) plasma. The above (I) and (II) shows the current response at a constant potential 0.8V, and the above (III) and (IV) monitors the current value at a constant potential 1.0V Therefore, the antibody-immobilized multi-array electrode is composed of a plurality of pairs of electrodes (working electrode and counter electrode), and each of the pair of electrodes is configured to detect a single target. That is, it is not a configuration in which a plurality types of targets are simultaneously detected by a single working electrode. Furthermore, the gold nanoparticles used as labels in Patent Document 3 do not have a current response.

As shown in FIG. 7B on Patent Document 4, there are two working electrodes, whereby two different types of antigens are simultaneously detected, and not two or more different types of antigens are simultaneously detected by a single working electrode. Also, the gold nanoparticles used for labeling do not respond to current.

Further, Patent Document 5 is to detect different sites in the same target substance as the first target and the second target, and is not configured to simultaneously detect different types of target substances.

An object of the present invention is to provide a detector and a method for detecting bacteria and/or viruses for capable of collectively detecting two or more types of bacteria and/or virus in a sample to be detected based on electrochemical or optical attributes unique to a label, and performing a quantitative and highly selective detection by using the label corresponding to each of various bacteria and/or virus species, for example, Enterobacteriaceae, Enterohemorrhagic Escherichia coli, and Staphylococcus aureus.

Another object of the present invention is to provide a label kit and a measurement kit used in the detector and the method for detecting bacteria and/or virus.

### Means for Solving the Problem

The present disclosure describes a bacteria and viruses detector including:
an electrode chip to which two or more types of metal nanostructures capable of specifically binding a specific target are attached;
a voltage applying unit for applying a voltage within a predetermined voltage range to the electrode chip;
a current measuring unit for measuring a peak current value output from the electrode chip according to the applied voltage;
a data storage unit for storing in advance the peak current value and the applied voltage value at the time of the peak current for each of the plurality of types of metal nanostructures;
a target specifying unit for identifying the target bound to the metal nanostructure by comparing a measured data of the current measuring unit and a stored data of the data storage unit; and
a display unit for displaying the identified target.

The target specifying unit may identify an estimated amount of a target bound to the metal nanostructure.

The “electrode chip to which two or more types of metal nanostructures are attached” means that two or more kinds of metal nanostructures may be attached to the electrode chip in advance, or two or more types of metal nanostructures may be attached to the electrode chip in case of measuring.

Another disclosure describes an electrode chip utilized in the bacteria and viruses detector including:
one or more electrodes formed by metal, carbon, or conductive glass, or electrodes formed by metal plating, or by printing using conductive ink.

The electrode is preferably a single electrode including a working electrode, a counter electrode, and a reference electrode.

The electrode chip utilized in the bacteria and viruses detector includes two or more types of metal nanoparticle structures that specifically bind to each of two or more types of targets. A first metal nanostructure for the first target, a second metal nanostructure for the second target, a third metal nanostructure for the third target, and an n^{th} metal nanostructure for the n^{th} target are each specifically bound. The “n” depends on the number of types of targets to be detected. This makes it possible to detect a plurality of different types of targets.

Another disclosure of a label kit for electrochemical detection may include two or more types of metal nanostructures having different current response and/or electrochemical properties from each other,
wherein a particular target can be specifically bound to the metal nanostructures, and the label kit identifiers the target from the electrochemical properties of binding bodies of the target and the metal nanostructures formed by mixing with a sample liquid containing at least one or more types of the targets.

Another disclosure of a label kit for electrochemical detection may include at least a first metal nanostructure capable of specifically binding to a first target and a second metal nanostructure capable of specifically binding to a second target, wherein the first metal nanostructure and the second metal nanostructure are mixed in a sample liquid containing at least the first target or the second target so that the biding bodies of the target and the metal nanostructure are formed and the target is identified from the electrochemical properties of the biding bodies.

The label kit for electrochemical detection may further include a third metal nanostructure that specifically binds to the third target, and an n^{th} metal nanostructure that specifically binds to the n^{th} target. The “n” depends on the number of types of targets to be detected.

Another disclosure describes a detection kit set for bacteria and/or viruses including:
two or more selected from [i] a bacteria and virus detector with or without an electrode chip as described above, [ii] an electrode chip for a bacteria and virus detector as described above, and [iii] a label kit for electrochemical detection as described above.

Another disclosure describes a detector for detecting bacteria and/or viruses including:
an attribute data detection unit for electrochemically or optically detecting attribute data of a metal nanostructure in a specific binding metal nanostructure from binding bodies which specifically bind to a target to the specific binding metal nanostructure,
wherein the binding bodies are obtained by contacting a label containing at least two or more types of the specific binding metal nanostructures each having a different attribute with a sample containing one or more types of targets;
a data memory unit for storing the attribute data of at least two or more types of the metal nanostructures and collation data including at least target data or label data associated with the attribute data; and
a target determination unit for determining a type of a target corresponding to the detected attribute data based on the attribute data detected by the attribute data detection unit and the collation data.
The target determination unit may determine a type of the target and an estimated amount of the target.

The label may include at least a first specific binding metal nanostructure including a first antibody or a first aptamer capable of specifically binding a first target, and a second specific binding metal nanostructure including second antibody or a second aptamer capable of specifically binding to a second target different from the first target, wherein the second specific binding metal nanostructure have an attribute different from that of the first specific binding metal nanostructure.

The label may further include third specific binding metal nanostructure including a third antibody or a third aptamer capable of specifically binding to a third target different from the first and the second targets, wherein the third specific binding metal nanostructure have an attribute different from that of the first and the second specific binding metal nanostructures.

The target is bacteria and/or virus.

In case that two or more types of targets are present in the sample, the two or more types of targets can be detected separately, and when one type of target is present in the sample, the one type of target is detected.

The attribute data detection unit may include:
a single electrode consisting of a working electrode, a counter electrode and a reference electrode;
a voltage application controller for applying a predetermined voltage according to differential pulse voltammetry measurement to the working electrode and the reference electrode; and
a current response measuring unit for determining a current peak (peak height) of a current response measured by applying a predetermined voltage and a potential (peak potential) at the time of the current peak.

In this configuration, two or more types of targets can be detected simultaneously by using a single electrode consisting of a working electrode, a counter electrode, and a reference electrode, and determining a peak potential at a current peak in differential pulse voltammetry measurement.

The attribute data detection unit may include an image analyzer for imaging the biding bodies and analyzing the color of the metal nanostructure in the specific binding metal nanostructure and/or the shape of the biding bodies from the imaged color image data.

The attribute data detection unit may include a wavelength measuring unit for measuring one or more wavelengths and/or spectra selected from the group consisting of absorption, fluorescence, and scattering of the specific binding metal nanostructures in the biding bodies.

The data memory unit may temporarily store the collation data. The data memory unit may acquire the collation data from an external apparatus. The data memory unit may be configured to be capable of updating the collation data.

The data memory unit may store various setting values and measurement parameters, for example a detector, an image analysis unit, and a wavelength measurement unit, in addition to the verification data.

Another disclosure describes a measurement kit utilized in detecting a target that is bacteria and/or viruses, including:
a first sample holding unit for being adhered two or more types of specific binding metal nanostructures having different attributes in state on physically separated, and/or
a second sample holding unit for being adhered two or more types of specific binding metal nanostructures to the same region in the case where two or more types of metal nanostructures having different attributes have insulating properties.

The two or more types of metal nanostructures adhered to the first sample holding unit may be physically separated from each other even when the metal nanostructures have an insulating property or when the metal nanostructures have no insulating property.

The first sample holding unit may include one or two or more selected from below [i] through [iv],
[i] an electrode chip for measuring a current response, wherein the two or more specific binding metal nanostructures are adhered in a state of being separated from each other on or around the working electrode,
[ii] a slide glass utilized in a microscope, wherein the two or more specific binding metal nanostructures are adhered in a state of being separated from each other in a region where sample is placed or around the region,
[iii] a cover glass utilized in a microscope, wherein the two or more specific binding metal nanostructures are adhered in a state of being separated from each other,
[iv] an optical cell for containing a sample, wherein the two or more singularly bonding metal nanostructures are adhered to the inner surface of the optical cell in a state of being separated from each other.

The second sample holding unit may include one or two or more selected from below [i] through [iv],
[i] an electrode chip for measuring a current response, wherein the two or more specific binding metal nanostructures are adhered in a state of being separated from each other on or around the working electrode,
[ii] a slide glass utilized in a microscope, wherein the two or more specific binding metal nanostructures are adhered in a state of being separated from each other in a region where sample is placed or around the region,
[iii] a cover glass utilized in a microscope, wherein the two or more specific binding metal nanostructures are adhered in a state of being separated from each other,
[iv] an optical cell for containing a sample, wherein the two or more singularly bonding metal nanostructures are adhered to the inner surface of the optical cell in a state of being separated from each other.

The measurement kit may further include a cleaning liquid container filled with the cleaning liquid and/or a measurement liquid container filled with the measurement liquid.

Another disclosure describes a label kit utilized in detecting a target that is bacteria and/or virus, including:
a single label-package filled with label solution including two or more specific binding metal nanostructures each having different attributes; and/or
two or more label-packages, wherein each of the two or more label-packages is filled with label solution including specific binding metal nanostructures having different attributes from each other.

The specific binding metal nanostructures utilized in the detector, the method for detecting, the measurement kit, and the label kit have the following characteristics.
The attribute data of the metal nanostructure or the specific binding metal nanostructure may be one or two or more selected from electrochemical data, metal nanostructure particle size, color, wavelength, absorption, fluorescence, and scattering.

The attribute data of the two or more types of metal nanostructures or the specific binding metal nanostructures may have different characteristics that can distinguish the type of the target.

It is preferable that the two or more types of metal nanostructures or the two or more types of specific binding metal nanostructures have a structure in which a local battery phenomenon is suppressed. As a configuration in which the local battery phenomenon is suppressed, for example, it is preferable that one or both of pairs forming the local battery have an insulating property. As an insulating property, it is preferable that a part of or all the metal nanoparticles or a part of or all the metal nanostructures are covered with an insulating film.

That is, in case that two or more types of metal nanostructures are used, the phenomenon may occur in which the metal dissolves due to a potential difference between metals and the target cannot be accurately measured, and it is preferable to use a metal nanostructure covered with an insulating film in order to suppress this phenomenon.

Examples of the insulating film include a polymer coating and an oxide film.

Among the two or more metal nanostructures, at least one metal nanostructure may include copper nanoparticles or one noble metal selected from chemically stable gold nanoparticles, palladium nanoparticles, silver nanoparticles, and platinum nanoparticles.

Among the two or more metal nanostructures, at least one metal nanostructure may include a polymer composite including copper nanoparticles or one noble metal selected from chemically stable gold nanoparticles, palladium nanoparticles, silver nanoparticles, and platinum nanoparticles.

Among the two or more metal nanostructures, at least one metal nanostructure may include a polymer composite including copper nanoparticles or one noble metal selected from chemically stable gold nanoparticles, palladium nanoparticles, silver nanoparticles, and platinum nanoparticles, and the other metal nanostructures may include metal nanoparticles different from the noble metal selected, or metal nanoparticles different from the copper nanoparticles selected.

Among the two or more metal nanostructures,
(a) at least one metal nanostructure is a polymer composite comprising one noble metal selected from chemically stable gold nanoparticles, palladium nanoparticles, silver nanoparticles, and platinum nanoparticles,
(b) other metal nanostructures different from the metal nanostructures selected in the above (a), comprises at least one selected from below (i) through (vi),
   (i) a polymer composite including metal nanoparticles different from the noble metal selected in the above (a),
   (ii) a polymer composite including a noble metal selected, within the noble metal, from the noble metal different from the noble metal selected in the above (a),
   (iii) metal nanoparticles different from the noble metal selected in the above (a),
   (iv) noble metal nanoparticles different from the noble metal selected in the above (a),
   (v) metal oxide nanoparticles, and
   (vi) metal nanoparticles covered with a metal oxide film.

Examples of combination of (a) and (b) include, but are not limited to, the following:
[i] Polymer composite including the gold nanoparticles as the above (a) and polymer composite including one type of nanoparticles selected from palladium (Pd), nickel (Ni), iron (Fe), zinc (Zn), cadmium, lead, gallium, arsenic, thallium, manganese and bismuth as (i) of the above (b).
[ii] Polymer composite including silver nanoparticles as the above (a), and polymer composite including platinum nanoparticles as (iii) of the above (b).
[iii] Polymer composite including gold nanoparticles as the above (a), and silver nanoparticles as (iv) of the above (b).
[iv] Polymer composite including gold nanoparticles as the above (a), and metal oxide selected from copper (I) (Cu₂O), copper oxide (II)(CuO), iron oxide (II)(FeO), iron oxide (II,III) (Fe₃O₄), iron oxide (III) (Fe₂O₃), and zinc oxide (ZnO) as (v) of the above (b). The metal oxide may be a polymer composite.
[v] Polymer composite including silver nanoparticles as the above (a), and one type of nanoparticles selected from palladium (Pd), nickel (Ni), iron (Fe), zinc (Zn), cadmium, lead, gallium, arsenic, thallium, manganese, and bismuth covered with a metal oxide film as (vi) of the above (b). The metal nanoparticles covered with the metal oxide film may be polymer composite.

The metal of the metal oxide film is not particularly limited, and may be selected from, for example, copper (Cu), nickel (Ni), iron (Fe), zinc (Zn), cadmium, lead, gallium, arsenic, thallium, manganese, and bismuth.

Among the two or more metal nanostructures,
(c) at least one metal nanostructure is polymer composite including copper nanoparticles,
(d) other metal nanostructures different from the metal nanostructures selected in the above (c), and may include one or two or more selected from below (i) through (vi),
   (i) polymer composite including metal nanoparticles different from the copper nanoparticles selected in the above (c),
   (ii) polymer composite including one type of noble metal selected from chemically stable gold nanoparticles, palladium nanoparticles, silver nanoparticles, and platinum nanoparticles,
   (iii) metal nanoparticles different from the copper nanoparticles selected in the above (c),
   (iv) metal oxide nanoparticles different from the copper nanoparticles selected in the above (c), and
   (v) metal nanoparticles covered with a metal oxide film different from the copper nanoparticle selected in the above (c).

Examples of combination of (a) and (b) include, but are not limited to, the following:
[i] Polymer composite including the copper nanoparticles as the above (c), polymer composite including one type of nanoparticles selected from palladium (Pd), nickel (Ni), iron (Fe), zinc (Zn), cadmium, lead, gallium, arsenic, thallium, manganese, and bismuth as (i) of the above (d).
[ii] Polymer composite including the copper nanoparticles as the above (c), polymer composite including one type of noble metal selected from gold nanoparticles, palladium nanoparticles, silver nanoparticles, and platinum nanoparticles as (ii) of the above (d).
[iii] Polymer composite including the copper nanoparticles as the above (c), polymer composite including one type of nanoparticles selected from palladium (Pd), nickel (Ni), iron (Fe), zinc (Zn), cadmium, lead, gallium, arsenic, thallium, manganese, and bismuth as (iii) of the above (d).
[iv] Polymer composite including the copper nanoparticles as the above (c), polymer composite including metallic oxide selected from copper oxide (I) (Cu₂O), copper oxide (II)(CuO), iron oxide (II)(FeO), iron oxide (II,III) (Fe₃O₄), iron oxide (III) (Fe₂O₃), and zinc oxide (ZnO) as (iv) of the above (d). The metal oxide may be a polymer composite.
[v] Polymer composite including the copper nanoparticles as the above (C), and nanoparticles coated with a metal oxide covering selected from palladium (Pd), nickel (Ni), iron (Fe), zinc (Zn), cadmium, lead, gallium, arsenic, thallium, manganese, and bismuth as (v) of the above (d). The metal nanoparticles covered with the metal oxide film may be polymer composite.

The metal of the metal oxide film is not particularly limited, and may be selected from, for example, copper (Cu), nickel (Ni), iron (Fe), zinc (Zn), cadmium, lead, gallium, arsenic, thallium, manganese, and bismuth.

The metal nanostructure may be one or more selected from:
a composite of AuNP (Au-nanoparticles) and PANI, a composite of PdNP (Pd-nanoparticles) and PANI, a composite of CuNP (Cu-nanoparticles) and PANI, a composite of AuNP and PmAP, a composite of AuNP and P o AP, a composite of AgNP (Ag-nanoparticles) and P o AB, a composite of AuNP and PmTD, AuNP small (NP_small size), AuNP large (NP_medium size), AuNP large (NP_large size), AgNP small (NP_small size), AgNP large (NP_medium size), AgNP large (NP_large size), Fe₂O₃NP(Fe₂O₃-nanoparticles), Cu₂ONP(Cu₂O-nanoparticles), SnNP(Sn-nanoparticles), PdNP(Pd-nanoparticles), ZnONP(ZnO-nanoparticles), CdSe/ZnSNP(CdSe/ZnS-nanoparticles), CdSe/ZnSNP (CdSe/ZnS-nanoparticles).

The size relation of the average particle diameter is AuNP small (NP_small)<AuNP medium (NP_medium)<AuNP large (NP_large).

The size relation of the average particle diameter is AgNP small (NP_small)<AgNP medium (NP_medium)<AgNP large (NP_large).

“NP” is an abbreviation for nanoparticle.

In the metal nanostructure, the relationship between the peak potentials of two or more metal nanostructures used together is that the absolute value of the difference between the peak potentials of all combinations is preferably equal to or greater than 0.08, more preferably equal to or greater than 0.1, even more preferably equal to or greater than 0.12, and particularly preferably equal to or greater than 0.16.

For example, the three types of metal nanostructures preferably satisfy the following relationship.

|first peak potential-second peak potential|≥0.05,

|first peak potential-third peak potential|≥0.05, and

|second peak potential-third peak potential|≥0.05

The peak potential of the metal nanostructure and the peak potential of the specific binding metal nanostructure are substantially the same, and the relationship is also established in the specific binding metal nanostructure. “| |” indicates an absolute value.

The target is preferable to include two or more types selected from Escherichia coli, Salmonella, Enterobacteriaceae, Staphylococcus aureus, norovirus, and influenza virus.

(Method for Producing Metal Nanostructure)

The present disclosure describes a method of producing a metal nanostructure, the method including:
a step of preparation of metal nanostructure in a composite including metal nanoparticle and polymer by an oxidation-reduction reaction in an aqueous solution.

The step of preparation may include a step of:
oxidizing a monomer of the conductive polymer in the aqueous solution by the metal ion as the same time as reducing the metal ion by the monomer of the conductive polymer, and by performing polymerization of the polymer at the same time or substantially the same time as the formation of the nanoparticles in the process in which the oxidation and reduction reactions proceed simultaneously or substantially simultaneously so that an aggregate in which the metal nanoparticles are dispersed in the conductive polymer is formed. The step of forming the aggregate may be a step of forming a raspberry-type aggregate.

The step of preparation may include a step of:
mixing the aqueous monomer solution into solution containing metal ion or solution containing metal complex. In the step of mixing, in parallel with the polymerization reaction (A) proceeding, the monomer is oxidized to the metal ion or the metal complex, and the metal ion or the metal complex is reduced to the monomer to proceed the production reaction (B) of the metal nanoparticles.

The step of mixing may be performed by mixing the aqueous monomer solution and/or solution containing the metal ion or solution containing metal complex at room temperature or at a temperature higher than room temperature. The above reactions (A) and (B) proceed even at room temperature, but by controlling the reaction temperature by heating, the reaction can be further accelerated and the reaction time can be shortened.

In the step of mixing, an aqueous monomer solution may be added while stirring the solution containing metal ion during mixing. Since the reaction proceeds uniformly in the aqueous solution by stirring, the particle size of the formed metal nanostructure can be controlled uniformly.

The step of mixing may include:
controlling the particle size of the metal nanostructure by changing the reaction time and the reaction temperature.

The step of preparation may include a step of:
controlling the particle size of the metal nanostructure by changing the concentration of the monomer aqueous solution, the solution containing metal ion or the solution containing metal complex.

The step of preparation may include a step of:
removing the unreacted material monomer or metal ion or metal complex, as distinguished from the metal nanostructures prepared in the step of mixing.

By the step of controlling the particle size and the step of removing, a metal nanostructure having a more uniform particle size can be produced.

Another disclosure describes a target determination detector including:
a target type determination unit that determines a type of a target corresponding to an attribute data by analyzing the attribute data of a metal nanostructure in a specific binding metal nanostructure obtained by measuring binding bodies of the target and the specific binding metal nanostructures obtained by contacting a label containing at least two or more specific binding metal nanostructures having different attributes with a sample containing one or more targets, wherein the specific binding metal nanostructure capable of specifically binding to a target.

The target type determination unit may determine the type of the target or the label by collating the attribute data with the collation data including at least the target data or the label data associated with the attribute data of the at least two or more types of metal nanostructures.

The target determination detector may include a data memory unit that stores the collation data. The data memory unit may be configured to temporarily store data.

The target determination detector may include a data acquisition unit that acquires attribute data of the metal nanostructure obtained by measuring the binding bodies.

Another disclosure describes a method of detecting bacteria and/or viruses, the method including the steps of:
forming binding bodies of specific binding metal nanostructures and targets, by contacting a sample including one or more targets with label including at least two or more specific binding metal nanostructures each having a different optical attribute, wherein the specific binding metal nanostructure capable of specifically binding to a target; and
observing an optical attribute of the specific binding metal nanostructure in the binding bodies by an optical detection unit.

The target is two or more types of Gram-negative and Gram-positive bacteria, the label is set to a specific binding metal nanostructure having different optical attributes, e.g., color and/or shape, for each of two or more gram-negative and gram-positive bacteria, in the step of observing, two or more types of gram-negative bacteria and gram-positive bacteria are distinguished from each other by observing different optical attributes.

Differences in color and shape make it easy to distinguish.

Another disclosure describes a method of detecting bacteria and/or viruses, the method including:
a target type determination step of determining a type of a target corresponding to attribution data of a metal nanostructure in a specific binding metal nanostructure obtained by measuring binding bodies of the target,
wherein the binding bodies are obtained by contacting a label containing at least two or more specific binding metal nanostructures having different attributes with a sample containing one or more targets, and the specific binding metal nanostructure is capable of specifically binding to the target.

The target type determination step may determine the type of the target or the label may be determined by collating the attribute data with the collation data including at least the target data or the label data associated with the attribute data of the at least two or more metal nanostructures.

The target type determination step may determine the type of the target or the label based on the peak potential obtained by measuring a current response when a predetermined voltage is applied to the binding bodies.

The target type determination step may determine an estimated amount of the target based on the peak height obtained by measuring a current response when a predetermined voltage is applied to the binding bodies.

The target type determination step may determine the type of target or label based on a color and/or shape obtained by analyzing the image data of the binding bodies.

The target type determination step may determine the type of target or label based on one or more wavelengths and/or spectra selected from absorption, fluorescence, and scattering for the binding bodies.

The target type determination step may include the steps of:
before determining the type of the target or the label,
determining a potential at a current peak from a current response when a predetermined voltage is applied to the binding bodies;
analyzing the color and/or the shape of the metal nanostructures from the image data of the binding bodies; and/or
measuring one or more wavelengths and/or spectra selected from absorption, fluorescence, and scattering for the metal nanostructures of the binding bodies.

Another disclosure of a computer-executable programs may perform each one of the steps of the method of detecting bacteria and/or viruses described above when executed by the at least one processor.

Another disclosure of a non-transitory computer-readable storage medium that stores computer-executable program, computer-executable program may perform each one of the steps of the method of detecting bacteria and/or viruses described above described above when executed by the at least one processor.

Another disclosure of information processing apparatus includes:
at least one processor; and
a memory (which may be the storage medium) in which a computer-executable program executed by the processor is stored;
the computer-executable programs may perform each one of the steps of the method of detecting bacteria and/or viruses described above when executed by the at least one processor.
The information processing apparatus is not particularly limited, and examples thereof include a smartphone, a tablet, a smart watch, a wearable computer, a personal computer, a server, and a cloud server, and a single or a plurality of combinations of information processing apparatus may be configured to be connectable by wired and/or wireless communication means.

The target determination detector, the bacteria and viruses detector, and the detector may include, for example, a dedicated circuit, firmware, a memory storing a computer-executable program and a processing command, a display, a bus, an input/output interface, a communication device, and the like.

[Effect]

According to the present invention, the following effects can be obtained.

(1) Metal nanostructures have high-current responsiveness and light-scattering properties, enabling highly sensitive measurement of targets (bacteria and viruses), eliminating the need for cell-culture and PCR, and speeding up testing.

(2) Because it is a simple method by electrochemical detection or optical detection, antibodies that selectively bind to targets can be used to accommodate a variety of targets and achieve high selectivity in the simultaneous detection of targets at the single cell and particle level or multiple targets.

(3) In testing, quantitation of the target is also possible.

(4) It is possible to deal not only with food poisoning causative bacteria and norovirus, but also with influenza virus and new type coronavirus.

(5) Since the present invention can be carried out quickly with a simple apparatus and the result can be confirmed, it can be used for on-site use other than the test room, primary inspection (screening), and the like. Examples of the onsite include a cooking shop such as a ship, an airplane, a spacecraft, and a restaurant, a food factory, a pharmaceutical factory, a quarantine station, a sampling place, an inspection institution, a medical institution, and a bedside diagnosis.

### BRIEF DESCRIPTION OF THE DRAWINGS

### Brief Description of the Drawings

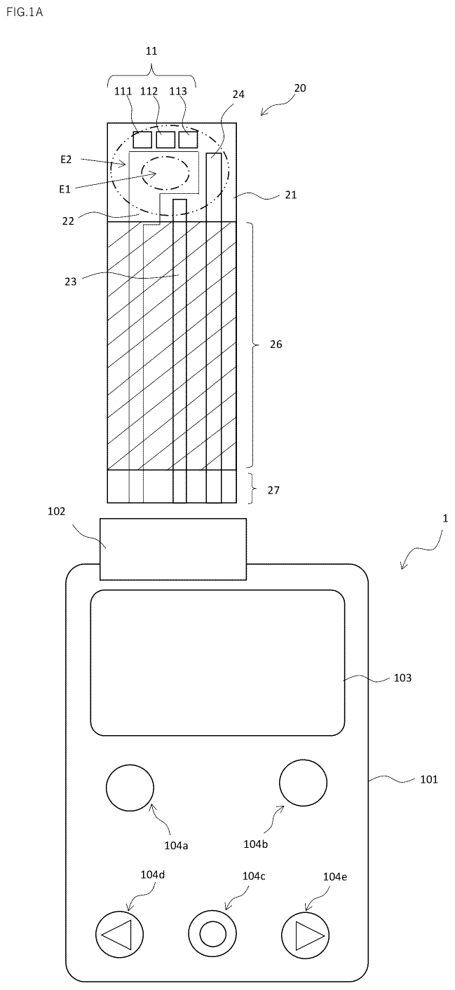

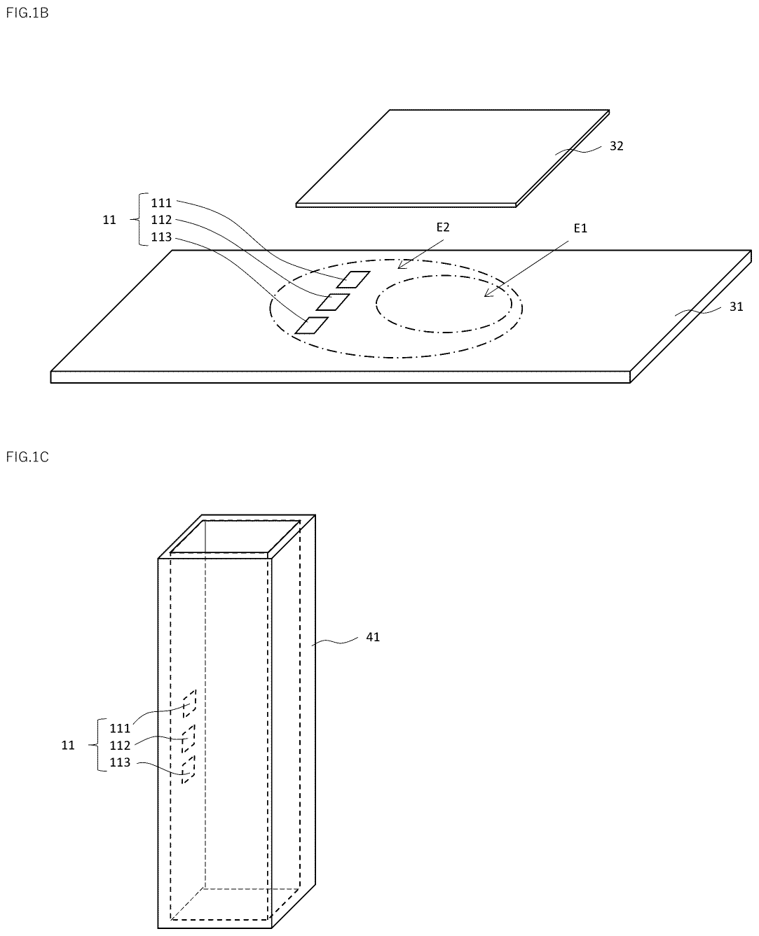

FIG. 1A is a diagram illustrating an exemplary electrode chip and DPV detector.

FIG. 1B is a diagram illustrating an example of a slide glass.

FIG. 1C is a diagram illustrating an example of an optical cell.

FIG. 2 is a diagram illustrating an example of a sample liquid container, a cleaning liquid container, and a measurement liquid container.

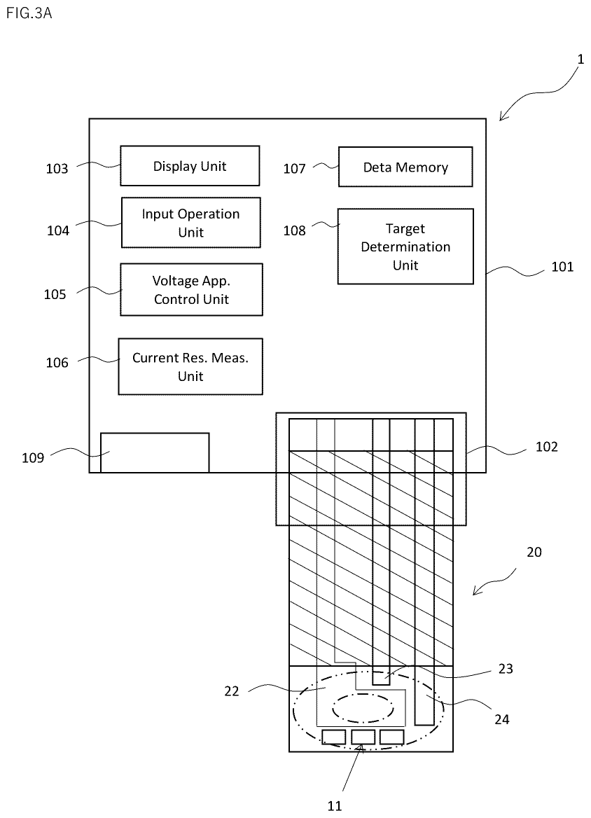

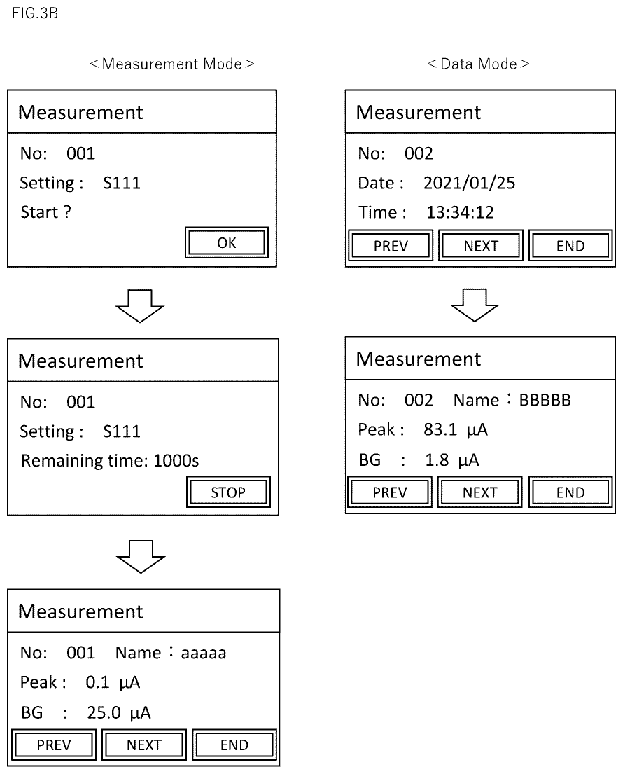

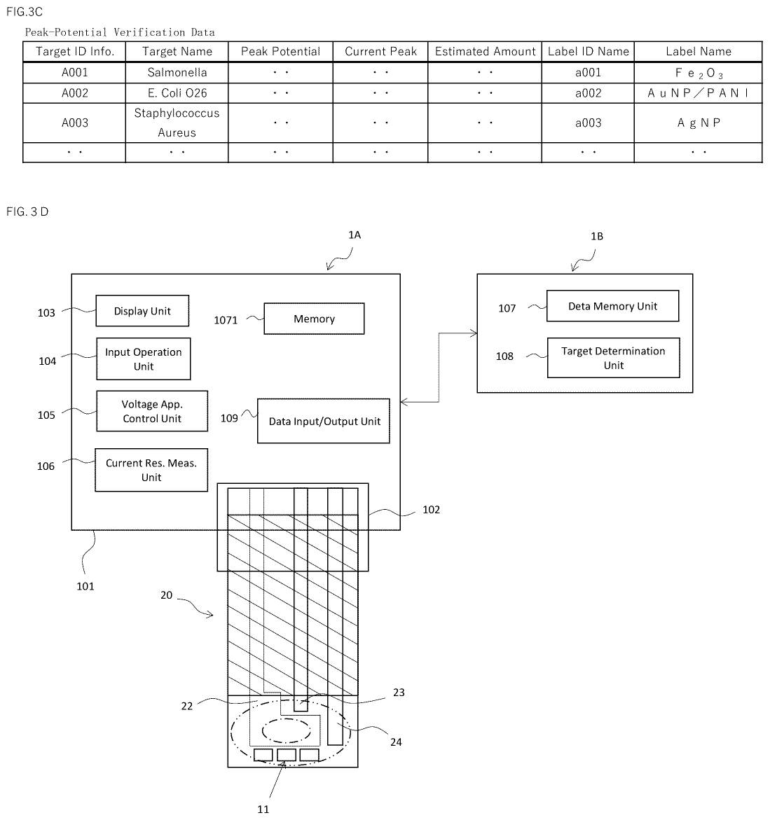

FIG. 3A is a functional block diagram for explaining an exemplary function of DPV detector.

FIG. 3B is a diagram illustrating an exemplary display of DPV detector.

FIG. 3C is a diagram showing an example of the peak potential verification data.

FIG. 3D is a functional block diagram of a DPV detecting system comprising a DPV detector and an information processing apparatus of another embodiment.

FIG. 4 is a functional block diagram for explaining an example of a function of an image analysis apparatus.

FIG. 5 is a diagram illustrating a functional block diagram for explaining an example of a function of a wavelength analysis device.

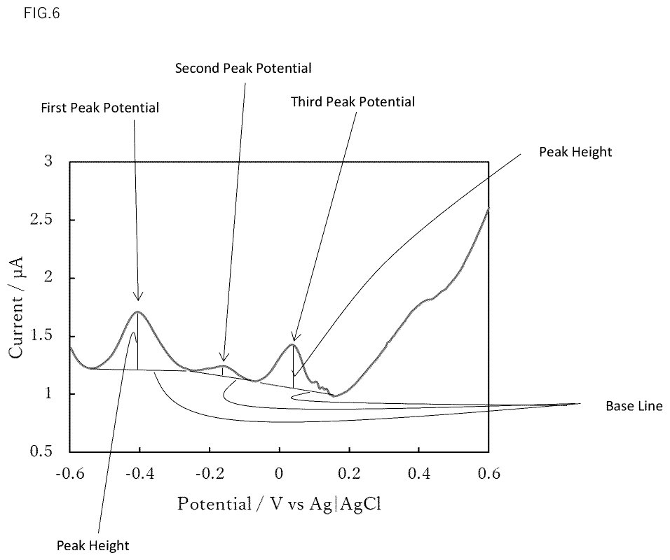

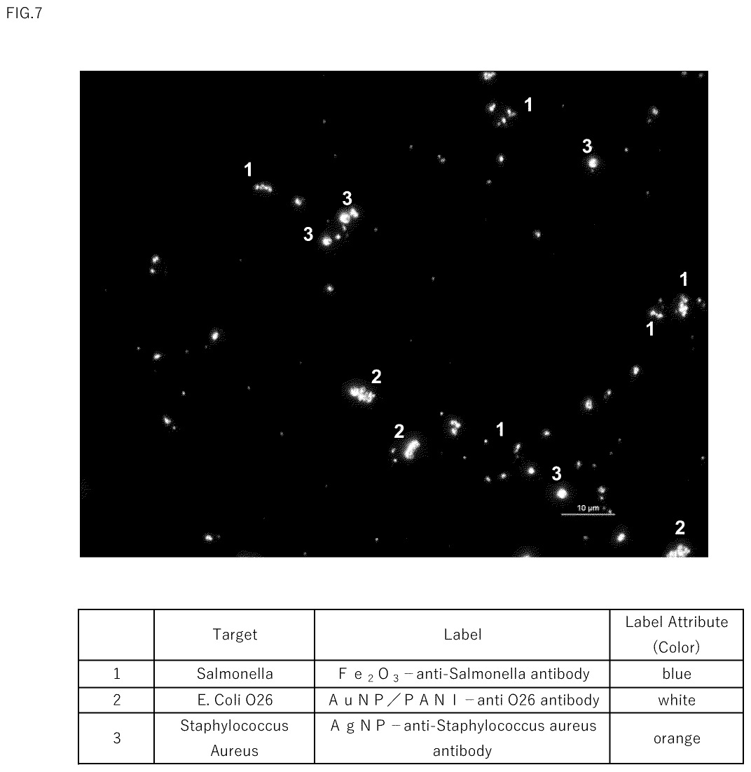

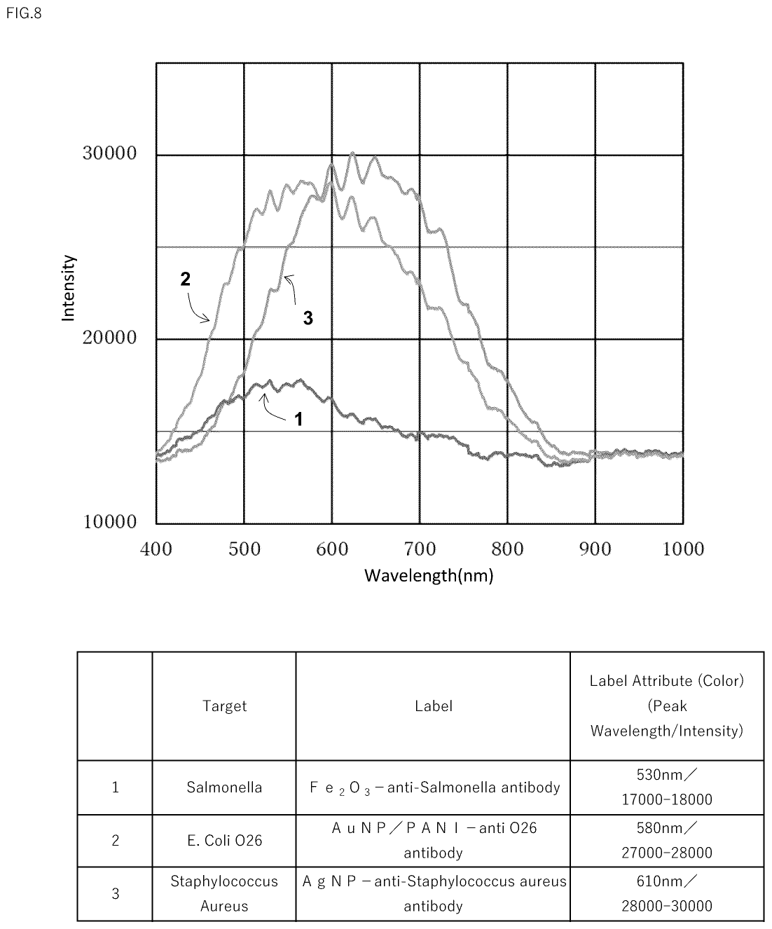

FIG. 6 is a diagram illustrating an exemplary current-response data measured by DPV

FIG. 7 is a diagram illustrating an example of a light spot of the shape of a cell (label-target binding bodies) imaged with a dark field microscope.

FIG. 8 is a diagram illustrating an example of a result detected by a scattered light spectrum measuring apparatus.

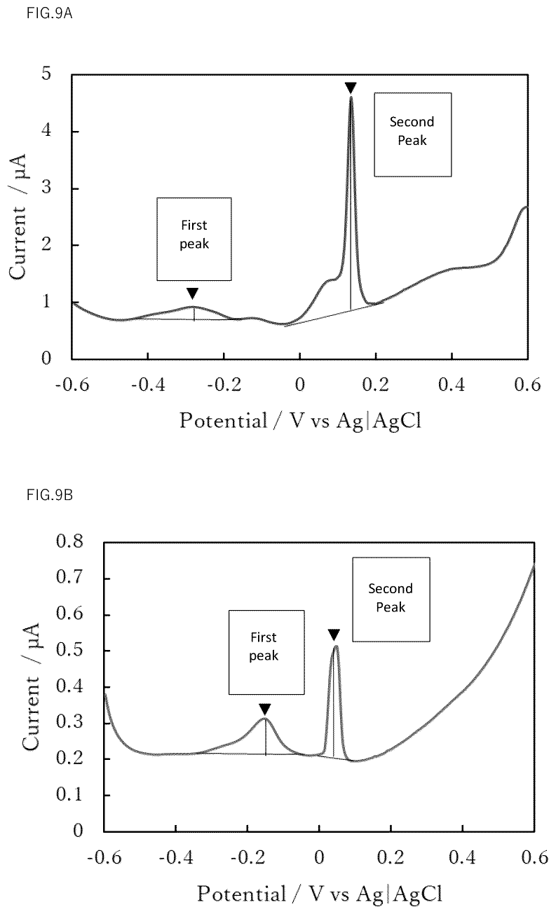

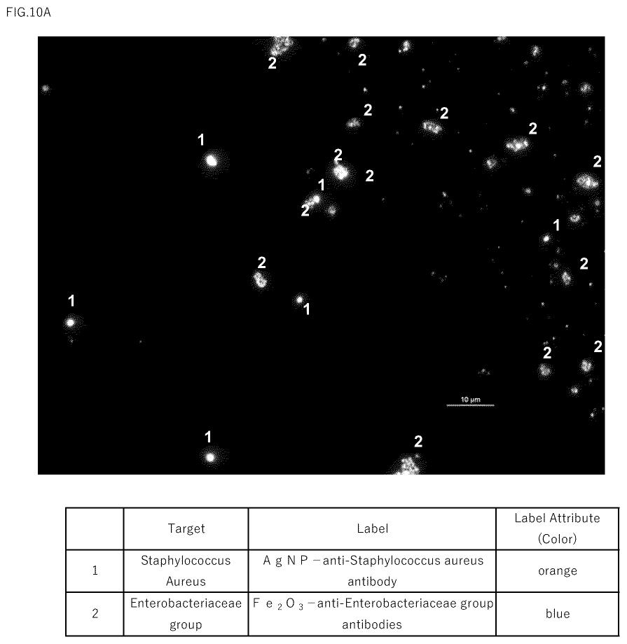

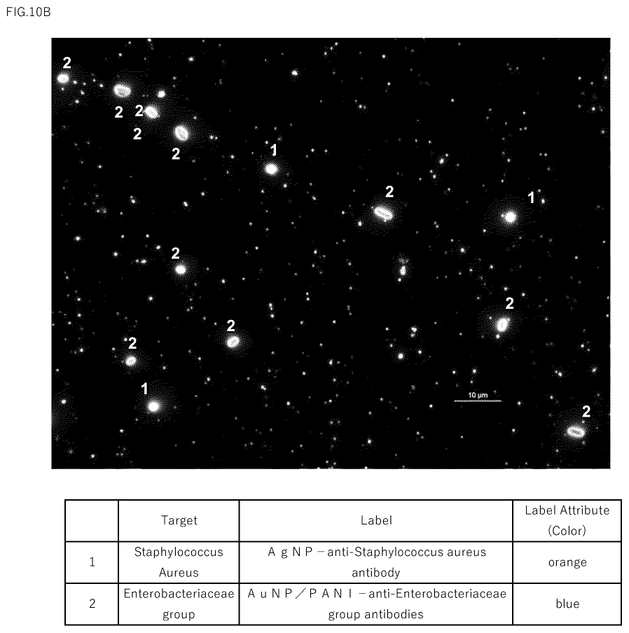

FIG. 9A is a diagram illustrating an exemplary current-response data measured by DPV

FIG. 9B is a diagram illustrating an exemplary current-response data measured by DPV

FIG. 10A is a diagram illustrating an example of a light spot of the shape of a cell (label-target binding bodies) imaged with a dark field microscope.

FIG. 10B is a diagram illustrating an example of a light spot of the shape of a cell (label-target binding bodies) imaged with a dark field microscope.

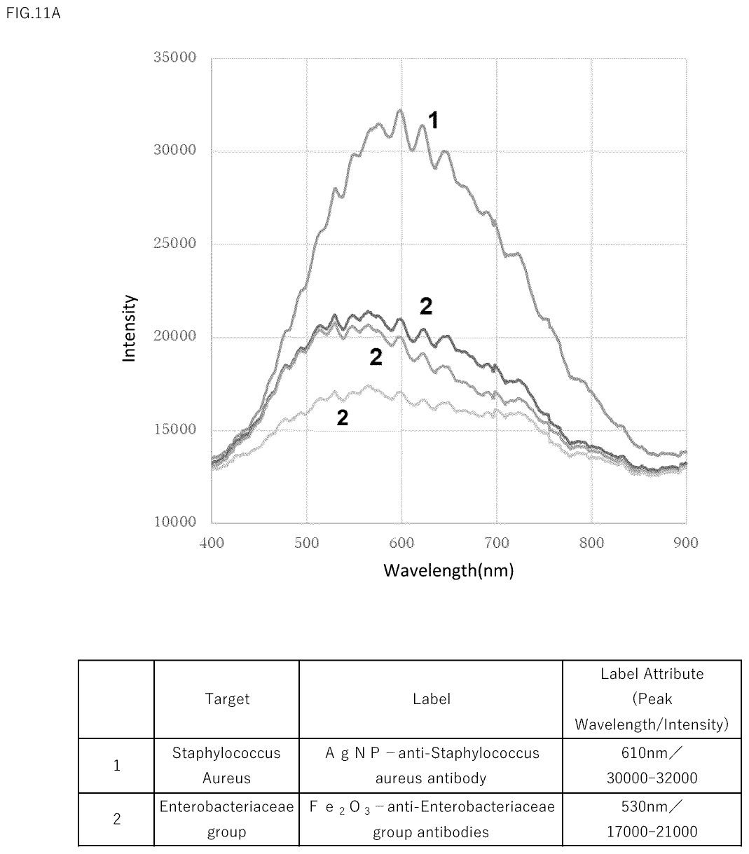

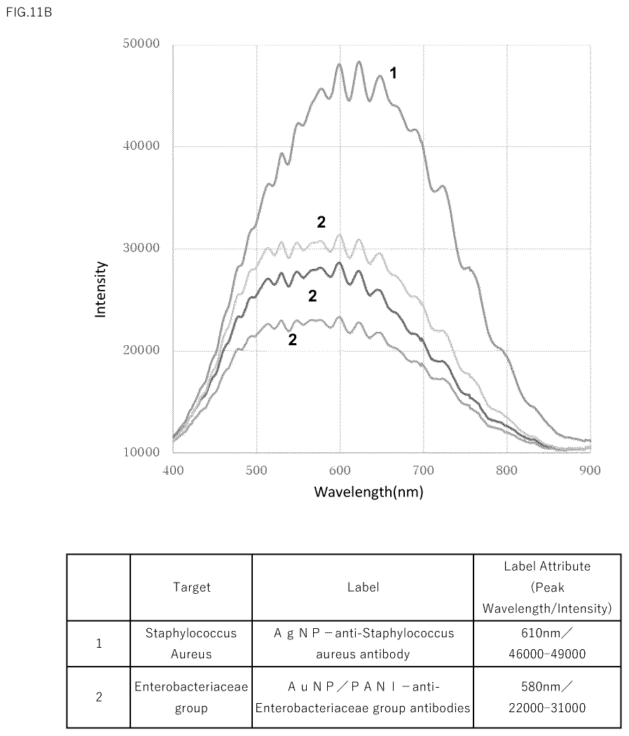

FIG. 11A is a diagram illustrating an example of a result detected by a scattered light spectrum measuring apparatus.

FIG. 11B is a diagram illustrating an example of a result detected by a scattered light spectrum measuring apparatus.

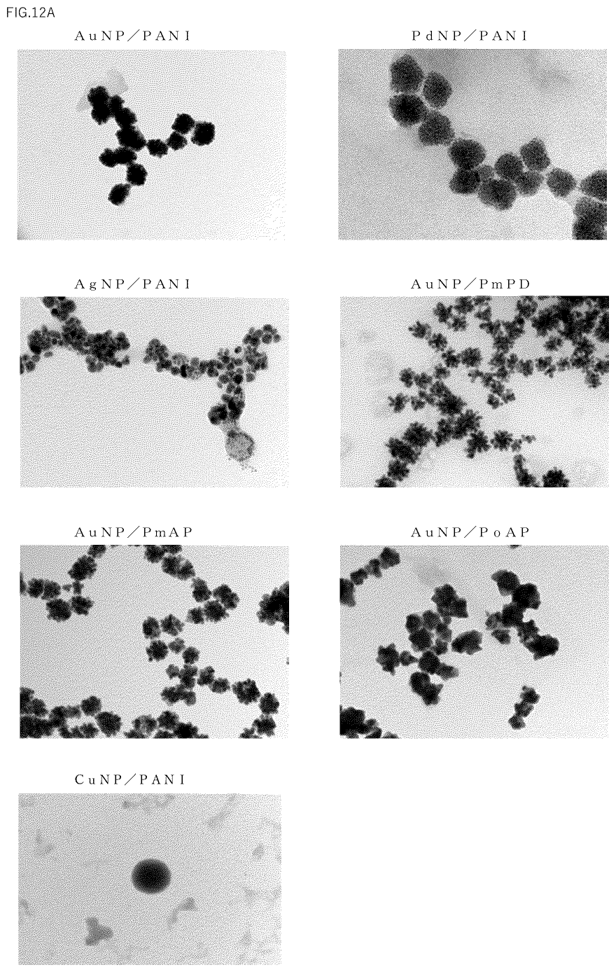

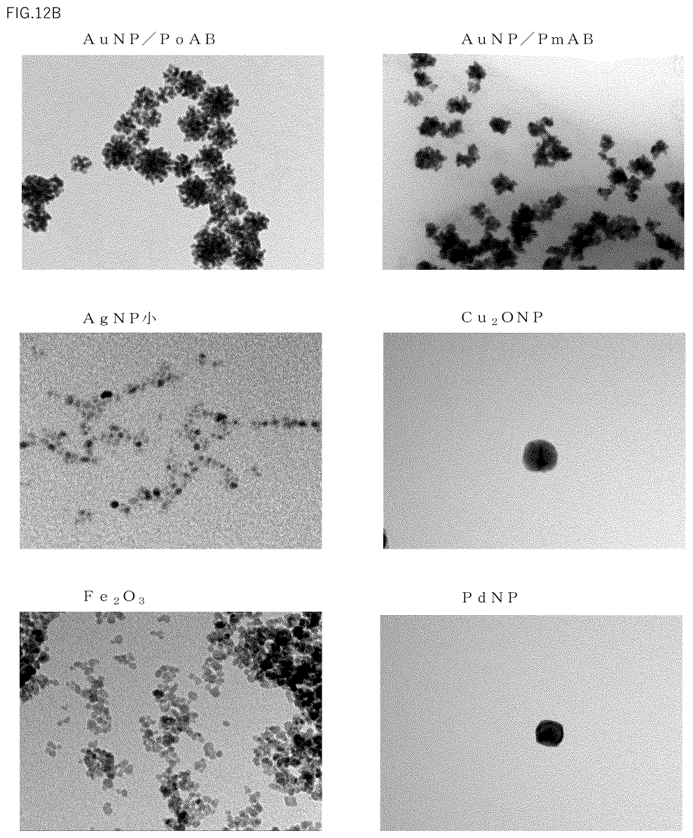

FIG. 12A is a diagram illustrating an example of imaging a label with an electron microscope.

FIG. 12B is a diagram illustrating an example of imaging a label with an electron microscope.

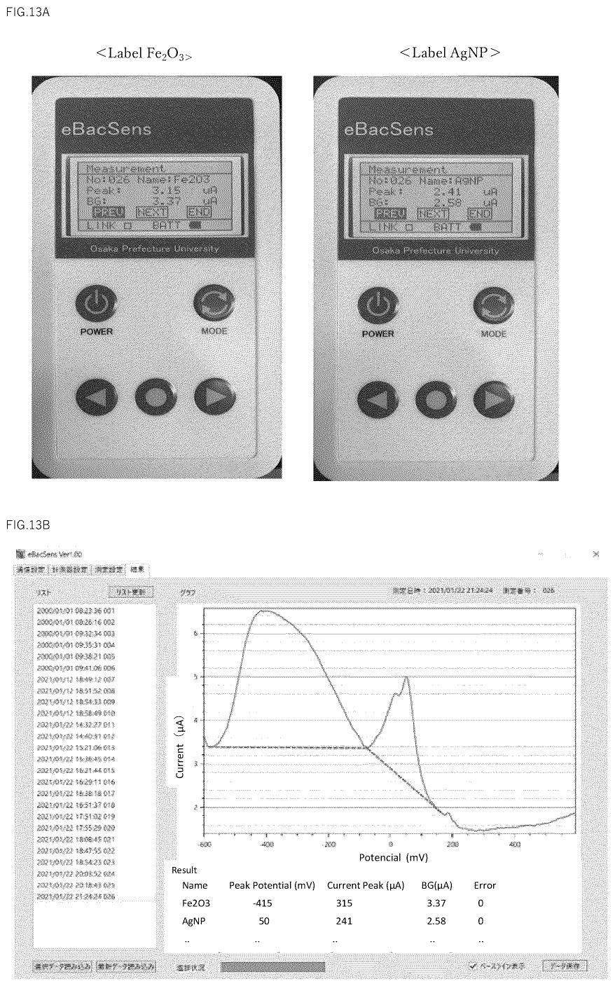

FIG. 13A is a diagram illustrating an exemplary measured result of DPV detector.

FIG. 13B is a diagram illustrating an exemplary current response-data measured by a DPV detector.

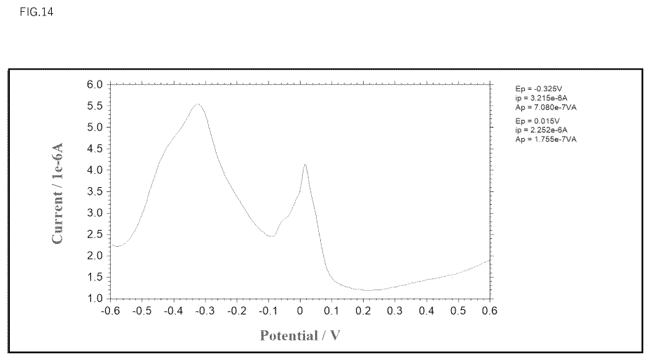

FIG. 14 is a diagram illustrating an exemplary current response-data measured by a DPV measuring device.

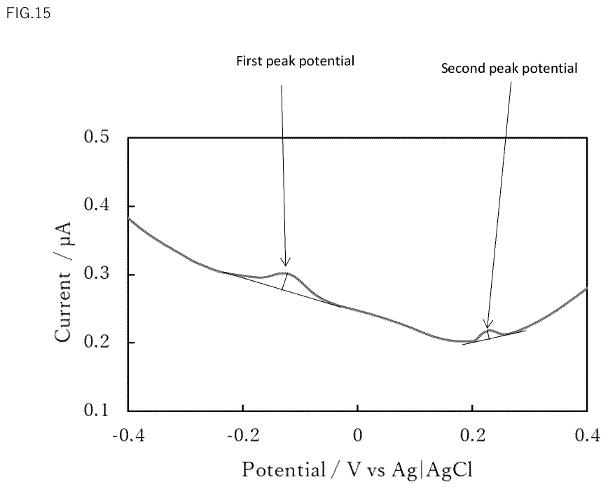

FIG. 15 is a diagram illustrating an exemplary current-response data obtained by DPV measuring a bacterium and a virus.

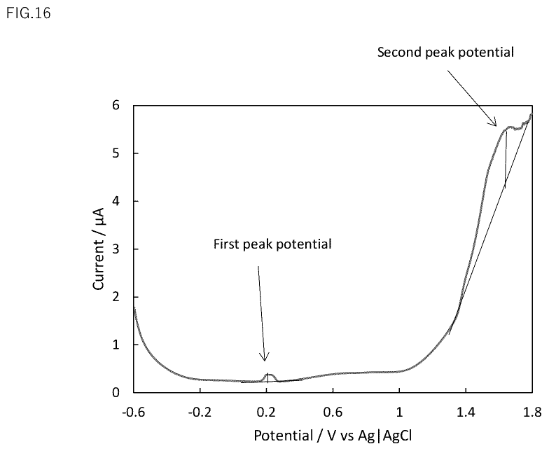

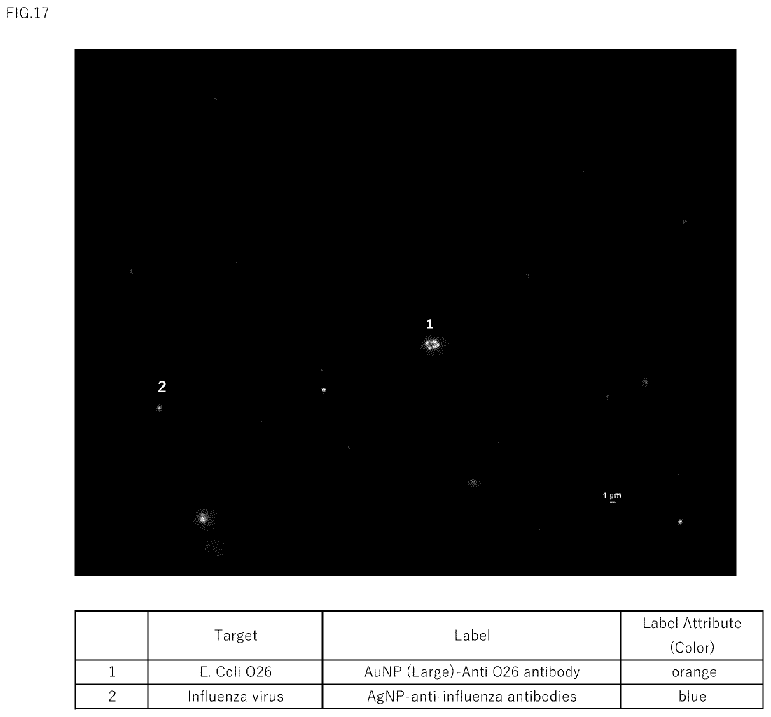

FIG. 16 is a diagram illustrating a current response-data DPV measuring two types of viruses.

FIG. 17 is a diagram illustrating an example of light spots of cells and virus shape (label-target conjugate) imaged with a dark field microscope.

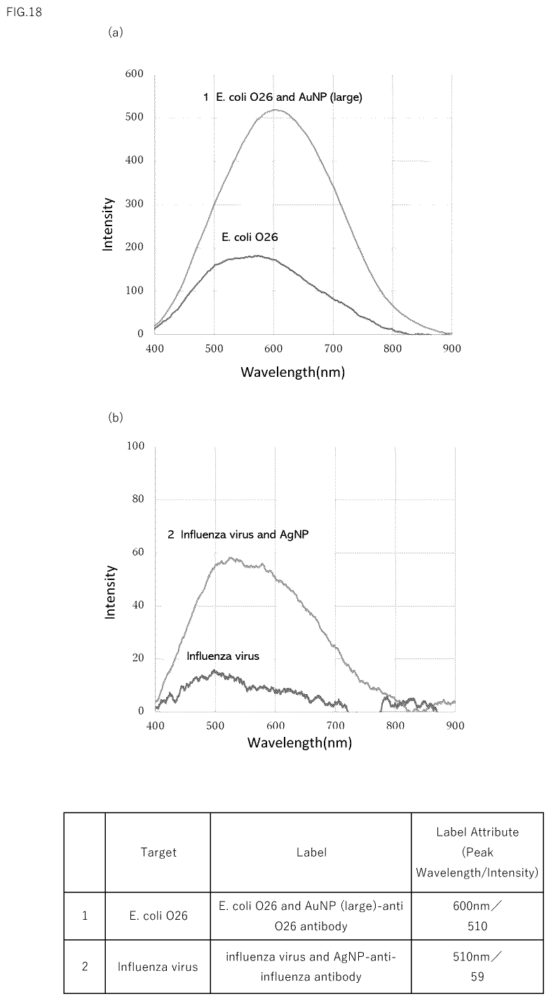

FIG. 18 is a diagram illustrating an example of a result detected by a scattered light spectrum measuring apparatus.

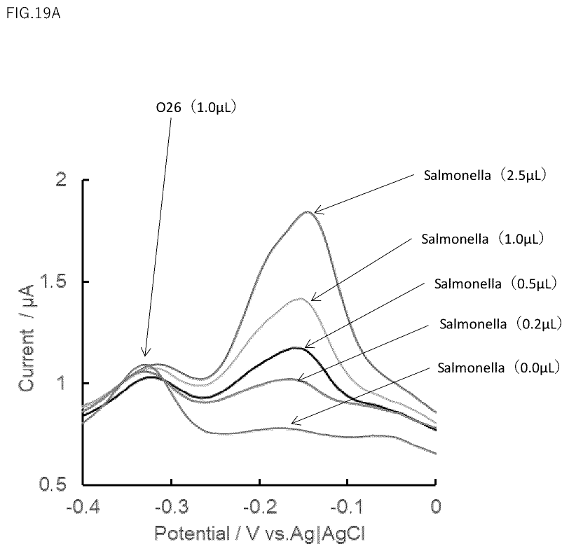

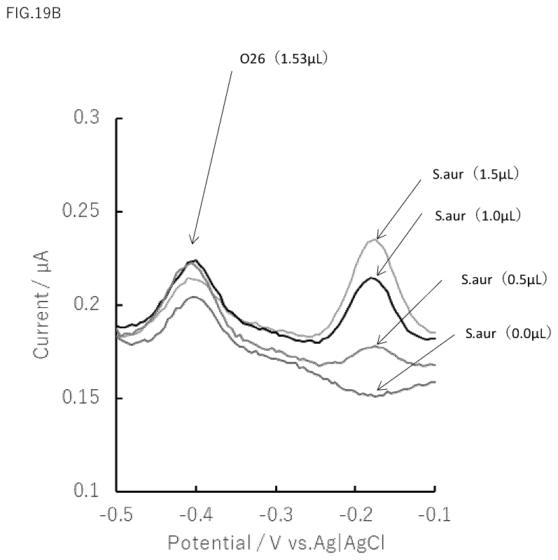

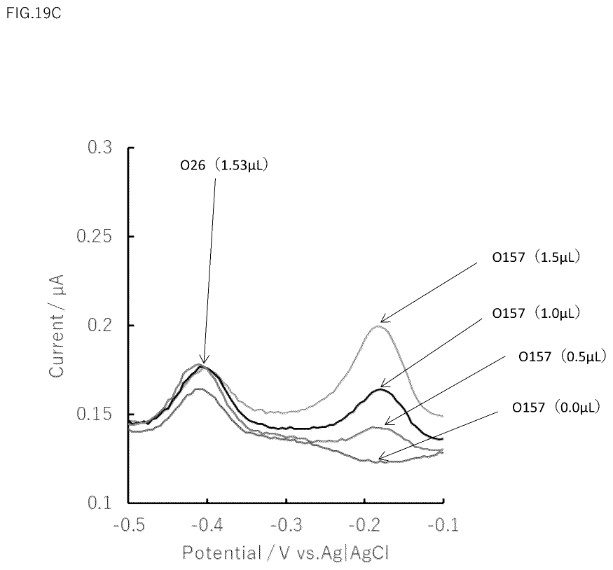

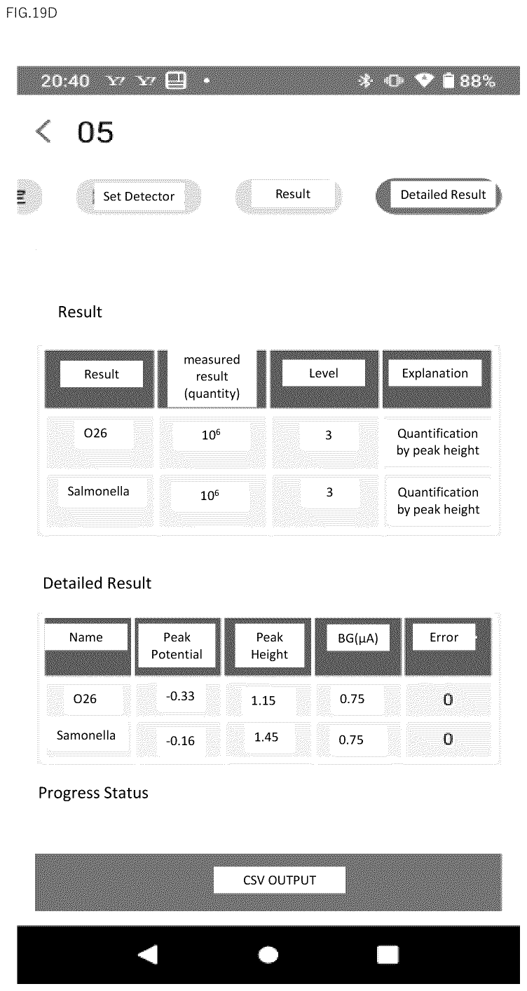

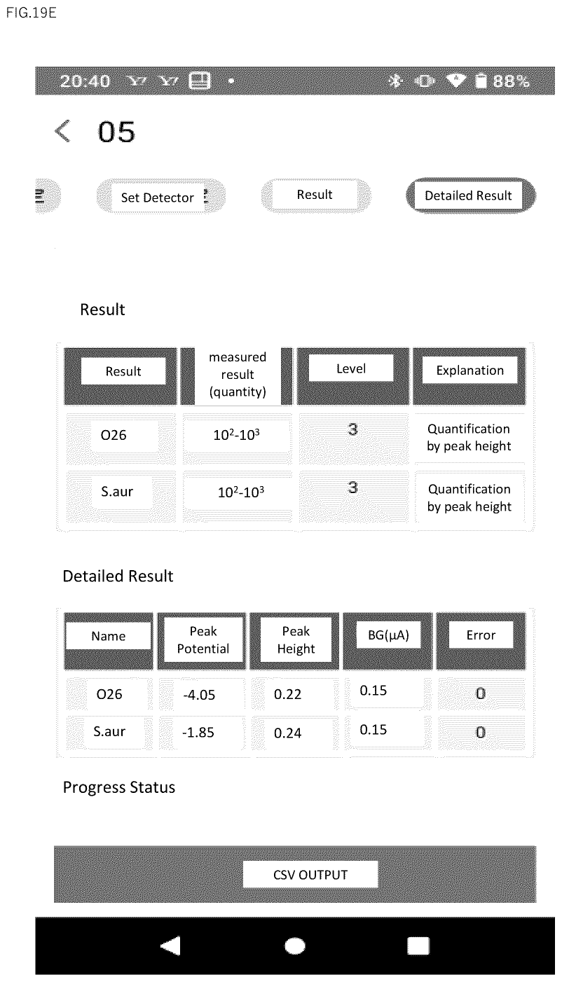

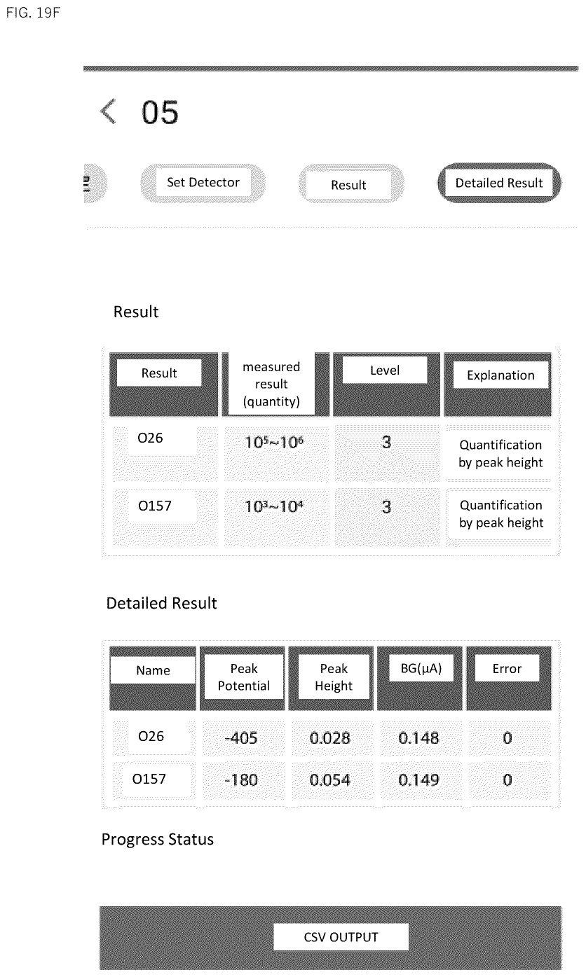

FIG. 19A is a diagram showing an example of the resulting can be quantified in DPV measured current-response data.

FIG. 19B is a diagram showing an example of the resulting can be quantified in DPV measured current-response data.

FIG. 19C is a diagram showing an example of the resulting can be quantified in DPV measured current-response data.

FIG. 19D is a diagram illustrating an example of a display screen showing an estimated amount (number of cells).

FIG. 19E is a diagram illustrating an example of a display screen showing an estimated amount (number of cells).

FIG. 19F is a diagram illustrating an example of a display screen showing an estimated amount (number of cells).

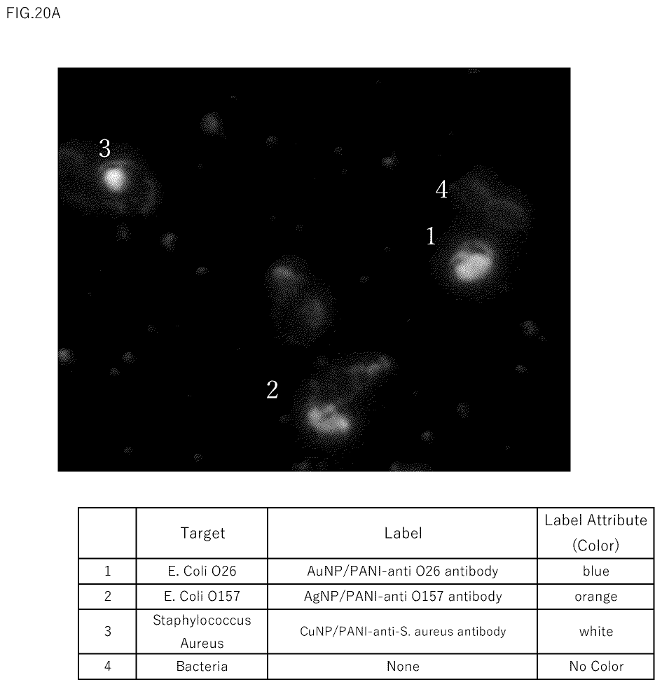

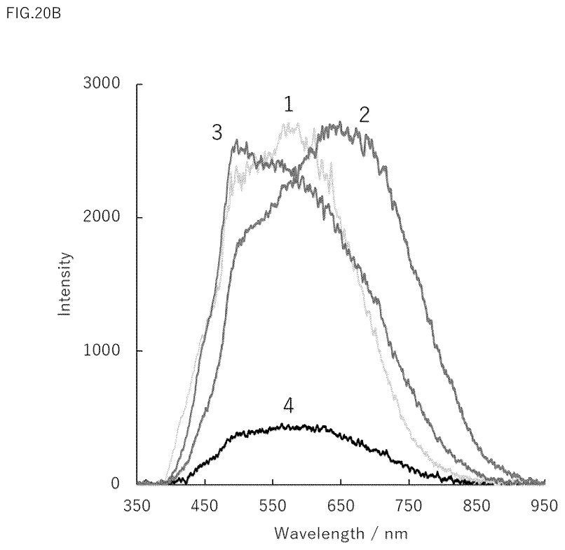

FIG. 20A is a diagram illustrating an example of a light spot of the shape of a cell (label-target conjugate) imaged with a dark field microscope.

FIG. 20B is a diagram illustrating an example of a result detected by a scattered light spectrum measuring apparatus.

### DETAILED DESCRIPTION

### Mode for Carrying Out the Invention

[Target (Sample)]

Targets include, for example, bacteria, viruses, bacterial groups, and the like.

Bacteria include, for example, E. coli, Salmonella, O 157, O 26, Staphylococcus aureus, and the like.

Examples of the virus include a norovirus, an influenza virus, and a coronavirus.

The Enterobacteriaceae group is Escherichia coli, Salmonella, and the like. Enterohemorrhagic Escherichia coli is O 157, O 26, etc.

In the present invention, as a target, a bacterial group including two or more kinds of bacteria can be detected as a single target. For example, as two types of targets, one type of first bacterium and a different type of bacterial group can be detected separately and simultaneously. As two targets, a first group of bacteria of one species and a specific bacterium of the first group of bacteria can be detected separately and simultaneously.

[Label: Metal Nanostructures]

The label is mixed with a sample liquid containing one or more targets to obtain a conjugate of the target and the metal nanostructures, and is used to identify the target from attribute data of the metal nanostructures in the conjugate.

The label preferably comprises at least two or more different current-responsive or optically-responsive metal nanostructures.

The metal nanostructure is, for example, a raspberry-like aggregate, nanoparticles or a nanoparticle-like aggregate. Aggregates are preferred because they may have better binding to the target than nanoparticles. In addition, the aggregate has a property of exhibiting a monochromatic color rather than nanoparticles, and in some cases, the aggregate is preferable for optical detection.

In the case of nanoparticles, each nanoparticle may have an outer layer formed by coating an organic substance.

The particle size of the individual nanoparticles constituting the metallic nanostructure is preferably from 1 nm to 100 nm, preferably from 1 nm to 60 nm, more preferably from 1 nm to 10 nm, and the specific surface area is preferably large.

In the present invention, by adopting a metal nanostructure having a large specific surface area, a sharp current peak can be formed, and detection can be performed by distinguishing a plurality of current peaks.

The metal nanostructures have immobilized antibodies or aptamers that specifically bind to the target. Details of the immobilization will be described later.

Examples of the metal nanoparticles constituting the metal nanostructure include gold (Au), silver (Ag), copper (Cu), copper oxide (I) (Cu₂O), copper oxide (II)(CuO), palladium (Pd), nickel (Ni), iron (Fe), iron oxide (II)(FeO), iron oxide (II,III), (Fe₃O₄), iron oxide (III), (Fe₂O₃), zinc (Zn), zinc oxide (ZnO), cadmium, lead, gallium, arsenic, thallium, manganese, and bismuth. In the case of multiple simultaneous detection, it is preferable to use two or more kinds of metal nanoparticles having different oxidation-reduction potentials.

According to the present disclosure, it is possible to detect a plurality of potentials (also referred to as “peak potentials”) at which the peak of the current appears from the measured data of the current response of the differential pulse voltammetry (DPV). That is, by selecting metal nanostructures having different peak potentials as labels, it is possible to simultaneously detect a plurality of types of targets.

Examples of the polymer constituting a part of the raspberry-like aggregate of the metal nanostructure include polyaniline (PANI), polypyrrole, poly 3,4-ethylenedioxythiophene (PEDOT), poly m-phenylenediamine (PmPD), poly m-aminophenol (PmAP), poly o-aminophenol (PoAP), poly m-aminobenzoic add (PmAB), poly o-aminobenzoic acid (PoAB), poly m-toluidine (PmTD), and derivatives thereof, which are conductive polymers. These conductive polymers are conductive in acidic solutions, but lose conductivity from neutral to alkaline and exhibit insulating properties.

The two or more metal nanostructures used at the same time in the measurement are selected in such a way that various non-measurability due to local battery phenomena is avoided.

In the present embodiment, in the two or more kinds of metal nanostructures, any one or two or more kinds of metal nanostructures may have an insulating property. A metal nanostructure having insulating properties and capable of measuring current response characteristics is selected.

For example, by covering one of the two metal nanoparticles capable of forming a local battery with an insulating coating, it becomes difficult to transfer electrons between the two metal nanoparticles, and it becomes possible to suppress dissolution of the other metal nanoparticles, so that suppression of formation of the local battery is realized.

The conductive polymer exhibits an insulating property in a neutral to alkaline region, but exhibits a current response even in an insulating property because oxidation or reduction reaction occurs at a predetermined potential. That is, the insulating coating covering the metal nanoparticles is not limited to the conductive polymer, and may be any compound that exhibits a current response by an oxidation reaction at a predetermined potential or a reduction reaction, and may be a polymer composed of a nucleic acid or an amino acid that generates an oxidation or reduction current such as DNA, peptide, or protein, or may be a polymer including a metal complex, or may be a metal oxide.

By appropriately using metal nanoparticles covered with an insulating coating in electrochemical detection, a plurality of kinds of metal nanoparticles can be used as labels. Different conductive polymers may be included for simultaneous detection of multiple targets.

The particle size of the raspberry-like metal nanostructures (also referred to as “composites”) is 10 nm to 200 nm, and the composite preferably has a structure in which ten or more metal nanoparticles having a particle size of 1 nm to 10 nm are contained in the polymer particles. Therefore, it is preferable that the thickness of the coating layers of the polymer is from 1 nm to 100 nm. That is, it is preferable that the polymer constituting the composite has a form from an oligomer to alow degree of polymerization (degree of polymerization of 100 or less). The degree of polymerization of the polymer in the complex depends on the oxidizing power of the metal ion. The particle size of the metal nanoparticles depends on the reducing power of the monomer. These can be used to control the particle size of the composite.

[Preparation of Raspberry-like Metal Nanostructures]

A metal nanostructure is prepared by a redox reaction in an aqueous solution.

For example, the monomer of the conductive polymer in the aqueous solution is oxidized by the metal ion, and the monomer of the conductive polymer reduces the metal ion. In the process of the simultaneous progress of the oxidation and reduction reactions, the polymer is polymerized at the same time as the nanoparticles are formed, and a raspberry-type aggregate in which the metal nanoparticles are dispersed in the conductive polymer is formed.

pH of the sample liquid containing the target is adjusted from the acidic region to the neutral region. Differential pulse voltammetry (DPV) measurements can be made in the acidic to neutral regions. Neutral regions are preferred because antibodies and metal nanoparticles are used. At this time, the conductive polymer contained in the raspberry-shaped metal nanostructure is insulating.

[Attributes of Metal Nanostructures]

The attribute data is selected from among electrochemical data such as current response, peak potential, metal nanostructure particle size, color, wavelength, absorption (wavelength, intensity, spectrum), fluorescence (wavelength, intensity, spectrum), scattering (wavelength, intensity, spectrum).

The attribute data has different characteristics so that different target types can be identified for simultaneous detection of multiple targets.

Among the two or more kinds of metal nanostructures, at least one kind of metal nanostructure may have insulating properties in a neutral solution.

Among the two or more metal nanostructures, at least one metal nanostructure may contain a chemically stable noble metal such as gold nanoparticles, palladium nanoparticles, silver nanoparticles, and platinum nanoparticles.

Among the two or more kinds of metal nanostructures, at least one kind of metal nanostructure may be a composite of a polymer containing the noble metal.

Among the two or more kinds of metal nanostructures, at least one kind of metal nanostructure may be a composite of a polymer containing the noble metal, and other metal nanostructures may contain metal nanoparticles other than the noble metal.

Among the two or more metal nanostructures, at least one metal nanostructure may be a composite of a polymer containing the noble metal, and other metal nanostructures may be a composite of a polymer containing metal nanoparticles other than the noble metal or a metal oxide nanoparticle, or a metal nanoparticle covered with a metal oxide film. Among the two or more kinds of metal nanostructures, it is preferable that all of other metal nanostructures except one have insulating properties, form a polymer film, or form metal oxide nanoparticles or metal nanoparticles coated with an oxide, thereby suppressing formation of a local battery between the two or more kinds of metal nanostructures under predetermined conditions (for example, when the metal nanostructures are in contact with a conductive material). As a result, two or more kinds of metal nanostructures may be brought into contact with each other for a long time, and distribution and storage in the same packaging container are also possible.

In addition to the same packaging container, it is possible to adopt a separate package that is physically separated until the time of use, and attachment to an electrode chip, a slide glass, or an optical cell may be conducted at a physically separated position.

In the case where a combination of metal nanostructures in which a local battery is formed under a predetermined condition is used in two or more kinds of metal nanostructures, it is preferable that the metal nanostructures be in a separate package that is physically separated from each other until the time of use, and it is preferable that the metal nanostructures be physically separated from each other even in a form in which the metal nanostructures are attached to an electrode chip, a slide glass, or an optical cell.

In the case of attaching the metal nanostructures to the electrode chip, the slide glass, or the like, the metal nanostructures may be attached so as to be opposed to each other across the dropping region of the sample liquid.

It is preferable that the label of the chemically stable metal nanostructure is contacted with the target (analyte) first when the local cell is formed, and then the label of the unstable or disappearing metal nanostructure is contacted with the target (analyte) when the local cell is formed.

[Specific Binding Metal Nanostructures]

The metal nanostructure is modified with an antibody or aptamer by immobilizing the binder to the metal nanostructure and binding the antibody or aptamer to the binder.

The antibody specifically binds a specific chemical structure of the target surface as an antigen, and may be an immune antibody obtained from immunity of an animal, an artificial antibody formed by a peptide or a synthetic polymer.

Aptamers include nucleic add molecules (RNA, DNA), peptides that specifically bind to a target.

The specific binding metal nanostructure i modified with the antibody is referred to as an antibody-modified metal nanostructure, and the specific binding metal nanostructure modified with the aptamer is sometimes referred to as an aptamer-modified metal nanostructure.

Examples of the binder include organic acids such as citric acid and ascorbic acid having a carboxy group, and salts thereof, sulfur-containing organic compounds having at least one functional group of a carboxy group, an amino group, and an amide group, or silicon-containing organic compounds, carbodiimide-based condensing agents represented by N-ethyl-N′-3-dimethylaminopropylcarbodiimide, biotin, avidin, and the like.

[Combination of Multiple Specific Binding Metal Nanostructures]

Depending on the different measurements, it is necessary to select different specific binding metal nanostructures in one or more attributes. Examples of combinations include:
(1) A combination of two or more specific binding metal nanostructures having different potentials at peak current
(2) Combination of two or more specific binding metal nanostructures having different colors
In this case, it is preferable to use a monochromatic aggregate as the metal nanostructure.
(3) Combination of two or more specific binding metal nanostructures having different shapes
(4) A combination of two or more specific binding metal nanostructures having different absorption wavelengths (peaks, spectra)
(5) A combination of two or more specific binding metal nanostructures having different fluorescence wavelengths (peaks, spectra)
(6) A combination of two or more specific binding metal nanostructures having different scattering wavelengths (peaks, spectra)
(7) A combination of two or more specific binding metal nanostructures having different antibody or aptamer for modification (the metal nanostructures are the same but the antibody or aptamer is different and the type of target can be determined from the shape of the target)
(8) In this combination, a label having a small current peak is used for bacteria or viruses contained in a large amount in a sample liquid, and a label having a large current peak is selected for bacteria or viruses which can be harmful to a human body even in a very small number
(9) A combination including two or more of the above (1) to (8).

Combinations of two specific binding metal nanostructures include, for example:
(1) Specific binding metal nanostructures including gold nanoparticles and specific binding metal nanostructures including noble metals including silver nanoparticles
(2) Specific binding metal nanostructures of gold nanoparticle-polymer composites and specific binding metal nanostructures including silver nanoparticles
(3) Specific binding metal nanostructures of silver nanoparticle-polymer composite and specific binding metal nanostructures including gold nanoparticles
(4) Specific binding metal nanostructures of copper nanoparticle-polymer composites and specific binding metal nanostructures including gold nanoparticles
(5) Specific binding metal nanostructures of copper nanoparticle-polymer composites and specific binding metal nanostructures including silver nanoparticles
(6) Specific binding metal nanostructures of gold nanoparticle-polymer composites and specific binding metal nanostructures including nanoparticles of copper, zinc, palladium, tin or iron
(7) Specific binding metal nanostructures of silver nanoparticle-polymer composite and specific binding metal nanostructures including copper, zinc, palladium, tin or iron nanoparticles
(8) Specific binding metal nanostructures of copper nanoparticle-polymer composites and specific binding metal nanostructures including gold, silver, zinc, palladium, tin or iron nanoparticles
(9) Specific binding metal nanostructures of metal nanoparticle-polymer composites composed of metals other than noble metals, and specific binding metal nanostructures of noble metals such as gold, silver, platinum or palladium

Combinations of three or more specific binding metal nanostructures include, for example:
(1) Specific binding metal nanostructures including gold nanoparticles, specific binding metal nanostructures including silver nanoparticles, and one or more selected from specific binding metal nanostructures including nanoparticles of copper, zinc, palladium, tin or iron
(2) Of the above (1), nanoparticles of a noble metal are composites coated with a polymer (3) Of the above (1), nanoparticles of a metal other than a noble metal are composites coated with a polymer
(4) Of the above (1), copper, zinc or iron is an oxide
(5) two or more selected from the group consisting of specific binding metal nanostructures including silver nanoparticles and specific binding metal nanostructures including nanoparticles of copper, zinc, palladium, tin or iron
(6) Of the above (5), silver nanoparticles are composites coated with a polymer
(7) Of the above (5), nanoparticles of copper or palladium are composites coated with a polymer
(8) Of the above (5), copper, zinc, or iron is an oxide
Gold nanoparticles have a peak potential of about 1.5V, and other metallic nanostructures other than gold may be used for electrochemical measurements due to water electrolysis, voltage-control by DPV methods, and shortened measurement times.

[Measurement Kit]

The measurement kit may include an analyte holding unit, a cleaning solution container, a measurement liquid container, a sample liquid container, a solvent or a dispersant for the sample liquid, and a label kit.

The analyte holder may be an electrode chip, a slide glass and a cover glass, an optical cell, or a conjugate pad. Two or more kinds of metal nanostructures having different attributes may be physically separated and attached to the analyte holding unit.

[Electrode Chip]

(1) The first electrode chip is an electrode chip for measuring a current response, and two or more kinds of labels that are specific binding metal nanostructures are attached to the electrode or in the vicinity of the electrode in a state of being spaced apart from each other. The electrode here may be one or two or more of the working electrodes, the reference electrode, and the counter electrode, but the working electrode is preferable.

The manner of attaching the label to the surface of the electrode chip is not particularly limited, and examples thereof include a method in which the label is dispersed in a solvent and dried on the surface of the chip, a method in which the label is printed and dried, a method in which the label is held on a conjugate pad such as a porous material, filter paper, a composite fiber, or a glass fiber, and the pad is fixed to the surface of the chip. Examples of the solvent include an aqueous solvent such as water, ion-exchanged water, pure water, and ultrapure water, and an aqueous solvent containing a water-soluble polymer.

The two or more labels move away from the surface of the electrode chip and bind to the target upon injection of the measurement liquid.

In FIG. 1A, the first electrode chip 20 is shown. The electrode chip 20 includes an insulating substrate 21, a working electrode 22 formed on the substrate 21, a reference electrode 23, and a counter electrode 24, an insulating coating portion 26 that protects the electrodes, a connection portion 27 that is electrically connected to the detection device, and a label 11 (a first label 111, a second label 112, and a third label 113) attached to the surface of the substrate 21 in the vicinity of the working electrode 22. A dotted line region E1 of the working electrode 22 indicates a dropping spot of the sample liquid (also referred to as an “analyte dropping region”), and a two-dotted line region E2 indicates a dropping spot of the measurement liquid. The measurement liquid is dropped so as to overlap with the sample liquid, and the label 11 moves away from the substrate 21 and binds to the target on the working electrode 22. Note that the measurement liquid is a solvent that plays a role of binding the label and the analyte (target).

In the analyte dropping area E1 of the working electrode 22, a predetermined surface roughness may be set in order to supplement the label, and a binding material that binds to the target may be immobilized.

FIG. 1 is a diagram illustrating an exemplary external view of a DPV detecting device in a lower part of a 1A. DPV detecting device 1 includes a housing, a connector connected to the electrode chip, a display unit, an input-operation unit, and the like. Details will be described later.

(2) The second electrode chip is an electrode chip for measuring the current response, wherein the label, which is one or more specific binding metal nanostructures, is not pre-attached on or in the vicinity of the electrode.

The electrode chip has a configuration in which no label is attached in advance, and one or more kinds of labels may be placed or attached to the electrode or the vicinity of the electrode in measuring, or a conjugate of the target and the label may be placed on the electrode.

The second electrode chip is a single electrode consisting of a working electrode, a reference electrode and a counter electrode. It can be connected to DPV detecting device 1.

(3) The third electrode chip is a multi-array electrode chip in which a plurality of pairs of electrodes including a working electrode and a counter electrode are arranged.

Each pair of electrodes is electrically non-contact with each other from other pair of electrodes, and the sample liquid injected into each pair of electrodes is physically and electrically non-contact with each other.

The third electrode chip may be used in a typical DPV measuring device.

(4) The first electrode chip may be a multi-array electrode chip in which a plurality of first electrode chips is arranged. The first electrode chip is electrically non-contact with other first electrodes to each other, and the sample liquid injected into each first electrode is physically and electrically non-contact with each other.

A label kit including two or more specific binding metallic nanostructures of the present disclosure may be used in a typical DPV measuring device using a multi-array electrode in which a plurality of pairs of electrodes consisting of a working electrode and a counter electrode are arranged.

Each of the electrodes described above may be formed of an electrode made of metal, carbon, conductive glass, or the like, or an electrode formed by using metal plating or printing a conductive ink.

At least the working electrode may be composed of a surface that has affinity to the target bacteria and virus (including the group) by immobilization of the binding material or formation of a microstructure.

The electrode chip of the present disclosure may be used by being attached to DPV detecting device 1, or may be used in a typical DPV measuring device. The electrode chips may be disposable and may be cleaned and reused.

In the surface layer of the electrode, a functional group or a binding site capable of forming an interaction or a bond with a target to be detected is preferably introduced (attached). The binding substance may be the functional group or the binding site.

The above functional groups and binding sites may be selected according to the detectable substance, for example, hydroxy group, amino group, imino group, carboxy group, carbonyl group, phosphoric acid group, sulfonyl group, thiol group, epoxy group, succinimide, hydrocarbon groups such as linear or branched aliphatic hydrocarbon groups, alicyclic hydrocarbon groups, aromatic hydrocarbon groups, or single-stranded DNA, RNA, aptamers, nucleic adds, immunoantibodies, artificial antibodies, enzymes, receptor such as proteins.

The functional group or the binding site can be introduced into the surface layer of the electrode by modifying a compound having a site capable of forming an interaction or a bond with the surface layer of the electrode and a site capable of forming an interaction or a bond with the object to be detected or the receptor capable of selectively binding the object to be detected on the surface of the electrode, or by mixing the compound with a conductive ink forming an electrode to form an electrode.

Interactions include, for example, hydrophilic-hydrophilic interactions, electrostatic interactions, and hydrophobic-hydrophobic interactions. Examples of the bonding include hydrogen bonding, metal-sulfur bonding, covalent bonding, and ionic bonding. Examples of the reaction include an antigen-antibody reaction, hybridization, and an enzyme reaction. The functional group or the binding site may be one kind or two or more kinds.

It is preferable that the surface-layer has irregularities and pores of 1 nm to 100 μm capable of forming an interaction or a bond with the object to be detected.

The irregularities and pores are appropriately selected according to the size of the object to be detected, and are preferably from 1 nm to 10 μm, and more preferably from 10 nm to 10 μm. The above-mentioned irregularities and holes efficiently cause the above-mentioned interaction and binding between the object to be detected and the surface of the electrode, and increase the contact area with the sample liquid containing the object to be detected (bacteria, viruses, etc.) to the electrode, or increase the adsorption amount by promoting adsorption of the object to the electrode, or shorten the adsorption time.

The surface roughness of the electrode may have, a square root height (Sq) or an arithmetic mean height (Sa) of 1 nm to 10 μm. Surface roughness is measured on a stylus-type surface roughness measuring machine (JIS B 0601(ISO4287) or on a non-contact measuring machine (according to ISO 25178) such as a white interferometer, a laser microscope, a digital microscope, a scanning probe microscope, etc.

The electrode may be formed of a conductive layer, such as a layer of metal or conductive ink, formed on the substrate by plating or screen printing.

Conductive layers can increase the contact area with the sample liquid containing the target to the electrode, increase the adsorption quantity by promoting the electrostatic adsorption, and shorten the adsorption time by physico-structural and chemical treatments such as surface microstructuring (100 nm-30 μm) and conformation (5 μm-100 μm).

The conductive ink is, for example, an ink containing a conductive material such as gold particles or gold nanoparticles, silver particles or silver nanoparticles, copper particles or copper nanoparticles, conductive carbon (100 nm to 30 micrometers), and the like.

By adding a surfactant to the conductive ink or applying a surfactant to the layer of the conductive ink as a chemical treatment in an electrode, in particular, a working electrode, the conductive ink layer can be hydrophilized. An electrostatic interaction or the like according to the zeta potential of the bacteria or virus particles as a target occurs. Thus, absorption of the target can be promoted. Examples of the surfactant include nonionic, cationic, anionic surfactants, fluorine-based surfactants, and silicone-based surfactants.

The blending ratio of the conductive ink and the surfactant is 0.1 mass % to 10.0 mass % of the surfactant with respect to 100 mass % of the conductive ink. The surfactant improves the absorptivity of bacteria and virus particles, for example, the adsorption amount and the adsorption time.

[Slide Glass and Cover Glass]

(1) The first slide glass is a slide glass used in a microscope, in which two or more specific binding metal nanostructures, or labels, are attached in a state of being spaced apart from each other. The first slide glass may or may not be attached with two or more labels separated from each other. Two or more kinds of labels may be attached to the first slide glass in a condition of being separated from each other, and the same two or more kinds of labels may be attached to the first cover glass in a state of being separated from each other, or one or more kinds of labels different from the labels attached to the first cover glass may be attached.

(2) The second slide glass is a slide glass used in a microscope, wherein the two or more specific binding metal nanostructures, or labels, are not attached in a spaced-apart manner. The second cover glass is attached with two or more labels spaced apart from each other.

The method of attaching the label to the surface of the slide glass or the surface of the cover glass is not particularly limited, and examples thereof include a method in which the label is dispersed in a solvent, dried on the surface of the glass, and dried after printing. Examples of the solvent include an aqueous solvent such as water, ion-exchanged water, pure water, and ultrapure water, and an aqueous solvent containing a water-soluble polymer.

The two or more labels are separated from the glass surface by contact with the sample liquid and bind to the target.

In FIG. 1B, the first slide glass 31, the label 11 (the first label 111, the second label 112, and the third label 113) attached to the surface of the first slide glass 31 are shown. The dotted line area E1 indicates a dropping spot of the sample liquid, and the two-dotted line area E2 indicates a dropping spot of the measurement liquid. The measurement liquid is dropped so as to overlap with the sample liquid, and is covered with a cover glass 32.

(3) The third slide glass and the third cover glass are not pre-attached with labels that are two or more specific binding metal nanostructures spaced apart from each other. When viewed under a microscope, one or more labels may be attached to the slide glass and/or the cover glass, and a conjugate of the target and the label may be placed on the slide glass.

[Optical Cell]

Optical cells are used in measuring each wavelength or spectrum, such as absorption, fluorescence, scattering, etc.

(1) The first optical cell is an optical cell that accommodate an analyte, the label being two or more specific binding metal nanostructures are attached to an inner surface thereof in a state of being spaced apart from each other. In FIG. 1C, the first optical cell 41, the label 11 (the first label 111, the second label 112, and the third label 113) attached to the inner surface of the optical cell are shown. The sample liquid is filled to a position above the label 11, and the label away from the inner surface binds to the target.

The manner of attaching the label to the inner surface of the first optical cell is not particularly limited, and examples thereof include a method of dispersing the label in a solvent and subjecting the label to drying, a method of printing and drying, and the like. Examples of the solvent include an aqueous solvent such as water, ion-exchanged water, pure water, and ultrapure water, and an aqueous solvent containing a water-soluble polymer.

The two or more labels are brought into contact with the sample liquid so as to move away from the inner surface and bind to the target.

(2) The second optical cell is not attached to its inner surface with labels that are the two or more specific binding metal nanostructures spaced apart from each other. In the measurement, one or more labels may be attached to the inner surface of the optical cell, or a sample liquid containing a conjugate of the target and the label may be loaded into the optical cell.

[Detection Liquid Container, Cleaning Liquid Container, Measurement Liquid Container]

FIG. 2 shows an analyte liquid container 51 filled with an analyte liquid, a cleaning liquid container 52 filled with a cleaning liquid, and a measurement liquid container 53 filled with a measurement liquid.

The analyte liquid (also referred to as “sample liquid”) contains one or more targets. The solvent or dispersant of the sample liquid is, for example, water, ion-exchanged water, or pure water. In the sample liquid, a food, a beverage, a vegetable, a meat, or the like containing a target may be dissolved or dispersed.

At the time of detection, the sample liquid is prepared by a predetermined procedure and is filled in the sample liquid container 51. The electrode chip 20 is inserted through the inlet 511, and the sample liquid is attached to the working electrode.

The cleaning liquid is used to remove, for example, a target, a foreign substance, or the like in an analyte that is attached to an electrode substrate or an electrode and is not required for measurement. Examples of the cleaning liquid include water, ion-exchanged water, pure water, ultrapure water, a buffer liquid, and physiological saline. The cleaning liquid is filled in the cleaning liquid container 52, and the electrode chip 20 is inserted through the inlet 521, and a substance that is not necessary for measurement in the sample liquid and is attached to the electrode chip 20 is removed from the electrode chip 20.

The measurement liquid is used in the electrochemical measurement. Examples of the measurement liquid include an electrolyte solution such as a phosphate buffer solution and a physiological saline solution. The measurement liquid is filled in the measurement liquid container 53, and the electrode chip 20 is inserted from the inlet 531 and brought into contact with the analyte and the label attached to the electrode chip 20. Contact with the measurement liquid separates the label from the electrode substrate and the label binds to the target attached to the working electrode.

Inlets 521, 531 may be sealed with an anti-reflux structure or a cap at the inlet 521, 531 to avoid discharge or drying of the content of the purifying or measurement liquid during the manufacturing and distribution process, and may be used by removing the sealing means at the time of use.

Further, as another embodiment, each of a solvent or a dispersing agent of the sample liquid, a cleaning liquid, and a measurement liquid may be individually packaged. These may be included in the measurement kit. At the time of detection, the individually packaged cleaning liquid may be filled in the cleaning liquid container and used, and the individually packaged measurement liquid may be filled in the measurement liquid container and used.

[Label Kit]

A single label package is used to detect a target that is a bacterium and/or a virus, and is filled with a label solution including two or more specific binding metal nanostructures each having different attributes.

In another embodiment, the plurality of label packages is each filled with a label solution including specific binding metal nanostructures having different attributes from each other.

The package is preferably a container that seals the contents and does not deteriorate the physical properties thereof, and examples thereof include a plastic bottle container, a film package container, and a glass container.

In the label kit, the labels may be packaged in a single measurement amount, and the labels may be packaged in bulk in quantities of several or more measurements. In addition, the label may be supplied in a form in which a required amount is attached to the electrode chip, the slide glass, or the optical cell. The form of the package is not particularly limited, and has a function of being safely transported and stored in a hygienic environment.

In the measurement, the label solution and the sample liquid are mixed, and the mixture is attached to an electrode chip or a slide glass, and electrochemical detection or optical detection can be performed. Alternatively, the sample liquid may be attached to the electrode chip or the slide glass, and then the label solution may be added dropwise, or vice versa.

[Detector: DPV Detector]

As shown in FIG. 3A, DPV detecting device 1 includes a housing 101, an electrode chip connecting substrate 102, a display unit 103, an input operation unit 104, a voltage application control unit 105, a current response measurement unit 106 (corresponding to a current measuring unit), a data memory unit 107 (corresponding to a data storage unit), a target determination unit 108 (corresponding to a target specifying unit or a target (type) determining unit), a data input/output unit 109, a power supply (not shown), and the like.

The electrode chip connecting substrate 102 is electrically connected to one end of the electrode of the electrode chip 20. The display unit 103 is, for example, a liquid crystal monitor or an organic EL monitor, and displays various settings, an input result by an input operation unit, and various target determination results. The input operation unit 104 is a user interface for input, and may be a touch panel that also serves as a liquid crystal monitor of the display unit 103, a push button, or the like. The data memory unit 107 may be a non-volatile memory or a volatile memory.

The input/operation unit 104 illustrated in FIG. 1A includes a power ON/OFF button 104a, a mode switch button 104b, an enter button 104c, a left cursor button 104d for moving the cursor, and a right cursor button 104e.

By operating the mode switch button 104b, various modes can be switched, for example, it is possible to select the measuring mode, data mode, maintenance mode, and the like. For example, the user can press the mode switch button 104b to move the cursor with the left/right cursor button 104d, 104e based on a command or information displayed on the display unit 103, and instruct a desired mode with the enter button 104c.

DPV detecting device 1 can detect two or more types of peak potentials. In order to detect two or more peak potentials, the current response characteristics of the labeled metal nanostructures are important factors. At the same time, the setting of the measuring parameters of DPV detecting device 1 is important.

The measurement parameters include the measurement range (current value range), starting and ending potential, measurement time, pulse amplitude, pulse width, pulse period, number of steps, base current sample time, Faraday current sample time, equilibrium potential and equilibrium time according to the number of detection, ΔE, and so on.

The setting of the measurement parameter may be incorporated into one of a plurality of modes, and the mode may be selected by a mode switch button 104b to set the measurement parameter.

The measurement parameters may be manually set, or data may be received via the data input/output unit 109 or a wireless communication unit (not shown) and stored in the data memory unit 107. A measuring parameter may be set by an information processing device (not shown) and sent to DPV detecting device 1.

When the electrode chip 20 to which the label 11 is attached in advance or the electrode chip 20 which is set together with the label 11 as a measurement kit, the measurement parameter or the identification information thereof is stored in a memory function (for example, a IC chip or a RFID chip) of the electrode chip 20, and when the electrode chip 20 is connected to the electrode chip connector 102, the measurement parameter or the identification information thereof may be transmitted to the data memory unit 107 and stored. In the data memory unit 107, the identification information and the measurement parameter thereof are stored in advance, and the measurement parameter corresponding to the read identification information may be set.

The measurement parameters corresponding to the plurality of targets may be stored in the data memory unit 107 as a database.

The measurement parameters (measurement range, starting and ending potentials, measurement time, pulse amplitude, pulse width, pulse period, number of steps, base current sample time, Faraday current sample time, equilibrium potential and equilibrium time according to the number of detections, ΔE, etc.) may be variably set corresponding to the label used. The detection accuracy can be maintained by changing the measurement parameters in accordance with the difference in the height of the current peak and the potential at the current peak for each label. These setting data may be input from the input operation unit 104, or various data of measurement parameters may be input from the data input/output unit 109.

The voltage application control unit 105 applies a predetermined constant voltage between the working electrode 22 and the counter electrode 24 of the electrode chip 20. The current response measurement unit 106 measures a current value corresponding to a constant voltage applied to the working electrode 22 and the counter electrode 24. If no current value is obtained, the target cannot be determined.

When the current value is obtained at the working electrode 22, the voltage application control unit 105 applies a voltage that repeats equilibrated and sweeping between the working electrode 22 and the reference electrode 23 according to the differential pulse voltammetry method. The current response measurement unit 106 measures the current value of the working electrode 22.

The current response measurement unit 106 detects the peak of the measured current. The potential at the peak of the detected current is referred to as a peak potential. The current response measurement unit 106 obtains a baseline by performing baseline fitting on the current measurement data, and detects a peak height and a BG based on the baseline.

When a peak of one current is detected, there is one type of binding body on the working electrode 22, and when a peak of a plurality of currents is detected, there are a plurality of types of binding bodies on the working electrode 22.

In addition, the current response measurement unit 106 may calculate the peak height of the peak of the measured current and the area of the peak curve of the current.

The data memory unit 107 stores peak potential matching data including at least two or more kinds of peak potential and data of two or more kinds of targets associated with the peak potential. The peak potential matching data may be stored in the data memory unit 107 in advance, may be received and stored via the data input/output unit 109 or a wireless communication unit (not shown), or may be updated at an appropriate timing.

In addition, the peak potential matching data may be stored in the data memory unit 107 when the peak potential matching data is stored in the memory function (IC chip, RFID chip) or the like of the electrode chip 20 and the electrode chip 20 is connected to the electrode chip connector 102.

In the present embodiment, the peak potential matching data may include target identification information, a target name, a current peak, a peak potential that is a potential at a current peak, a current peak area, label identification information, and a label name. FIG. 3C shows an example of the peak potential matching data.

The peak potential matching data may include various data such as a current peak (peak height), a peak potential, a type of a specific binding metal nanostructure, a type of a target that specifically binds to the specific binding metal nanostructure, and an estimated amount (quantitative value) of the target. The estimator may be an estimator associated with a current peak.

The target determination unit 108 compares the peak potential matching data with one or two or more kinds of peak potentials (and peak heights of the currents) detected by the current response measurement unit 106, and determines one or more kinds of targets or labels.

In addition, the target determination unit 108 may calculate an estimated amount of the target from a preset current response value per single cell or virus particle and a current peak (for example, a value of a peak height and an area (integrated value) of the current response). The “current response value per single cell or virus particle” is the same as the current response value per unit area. The “estimated amount of the target” is, for example, an estimated amount of the number of cells and the number of virus particles.

The target determination unit 108 may apply the peak potential and the current peak to data of the current response value range per preset cell number or virus particle number range, and display the type and estimated amount of the target or the label on an order of digits.

The display unit 103 displays the target determined by the target determination unit 108. Further, the display unit 103 may display an estimated amount of the target.

In addition, the display unit 103 may display one or more types of data among the current peak, the peak potential at the time of the current peak, the determined label and target, the current response value per single cell or per virus particle, and the estimated amount of the target.

In addition, when the numerical width of the plurality of peak potentials is larger than the predetermined threshold value, the display unit 103 may display the numerical width of the plurality of peak potentials smaller than the difference between the actual numerical widths in the graph display, and may indicate the peak height smaller. In the automatic display function of the display unit, when a small peak and a large peak are displayed, even a small peak becomes larger than the actual one, so that it is easy to visually recognize.

(Measurement Mode Description)

(1) The user attaches the sample liquid to the working electrode 22, and attaches the electrode chip 20 in a state where the measurement liquid is dropped to the electrode chip connecting substrate 102. After attachment, an operation of dropping the sample liquid and the measurement liquid onto the electrode chip 20 may be performed.

(2) Press the power ON/OFF button 104a and use the mode switching button 140b to move to the measuring mode and then use the enter button 104c to instruct.

FIG. 3B illustrates an exemplary screen-transition of the display unit 103. The display unit 103 displays the “measurement-start-waiting screen” in FIG. 3B of the drawing. The measurement number (identification number; No:001) and setting data (Setting: S111) are displayed. The setting information indicates identification information (name S111, etc.) of the measurement parameter (measurement time, pulse amplitude, pulse width, pulse period, number of steps, etc. used in differential pulse voltammetry). It should be noted that a configuration may be adopted in which a plurality of measurement parameters stored in the data memory unit 107 can be read out and selected by pressing the enter button 104c at the position of “Setting” after moving the cursor. Alternatively, a configuration may be adopted in which a selection is made for each item of measurement parameters.

(3) After moving the cursor to “OK”, instruct by the enter button 104c.

(4) The remaining measurement time (Remaining time: 1000 s) (counts down the preset measurement time) is displayed. When “STOP” is specified with the enter button 104c, the measurement is stopped.

(5) If there is no error indication (interruption) until the measurement time ends, the measurement is completed and the current response data is stored in the memory. The memory may be the data memory unit 107 or another built-in memory. The measurement date and the measurement time are also stored in association with each other.

(6) After the measurement is completed, the measurement number, the target name or the label name (Name: aaaaa) determined by the target determination unit 108, the peak-maximum value (Peak) of the current calculated by the current response measurement unit 106, and BG value (BG) are displayed on the display unit 103.

(7) In the display unit 103, when the cursor is moved and “PREV” is instructed by the enter button 104c, the previous result (target name or label name (Name), peak current value (Peak), and BG value (BG)) detected at the same time is displayed. In the display unit 103, when the cursor is moved and the “NEXT” is instructed by the enter button 104c, the result (target name or label name (Name), peak current value (Peak), and BG value (BG)) after the simultaneous detection is displayed.

After moving the cursor, when “END” is indicated by the enter button 104c, the display changes to the “Measurement-Start-Waiting” window.

Note that the measurement parameter and the peak potential matching data may be configured such that DPV detection device 1 and an information processing device (not shown) are connected to each other, various measurement parameters and peak potential matching data are inputted by the information processing device or stored in advance in a memory, and the data is sent from the information processing device to DPV detection device 1 and stored in the data memory unit 107 or the built-in memory.

(Data Mode Description)

(1) Check data measured in the past. Press the mode switch button 104b to enter the data mode and press the enter button 104c. When the mode shifts from the other mode to the data mode, the display unit 103 displays the “measurement number selection window” in FIG. 3B of the drawing, and displays the last measured data. The last measured data is, for example, a measurement number (No), a measurement date (Date), a measurement time (Time), or the like. When the cursor is moved in the “measurement number selection window” and “PREV” is instructed by the enter button 104c, the previous measurement number is entered, and when the cursor is moved and “NEXT” is instructed by the enter button 104c, the next measurement number is entered.

(2) When moving the cursor on the “measurement number selection window” and “ENTER” is instructed by the enter button 104c, the display go to the “setup name and measurement error window” of the selected measurement number.

(3) In the “setup name and measurement error window”, when the cursor is moved, and “ENTER” is instructed by the enter button 104c, the result of the measurement target of the selected measurement is displayed. The results displayed are, for example, a target name, a peak current value, a BG value, etc. When the cursor is moved, and “END” is instructed by the enter button 104c, the display returns to the “setup name and measurement error window”. If you move the cursor on this window, and instruct “END” by the enter button 104c, the display returns to the “measurement number selection window”.

(Maintenance Mode Description)

On the maintenance mode window, it is possible to set the date and time, delete the measurement data, and set the device-specific identification name.

### Another Embodiment

The display unit 103 may be configured to display one or more of the following data: the detected peak potential, the current value (peak height) at the peak potential, the determined label and target, the current response value per single cell (per unit area), and the estimated amount of the target (the number of cells and the number of virus particles).

DPV detecting device 1 may comprise a communication means and/or a communication interface for transmitting one or more of the data, the detected peak potential, the current value at the peak potential, the determined label and target, the current response value per single cell or viral particle (per unit area), or the estimated amount of the target, to an external device, and/or a recording means and/or a communication interface for storing the data on a recording medium. The data input/output unit 109 may also serve as a function thereof. Examples of the external device include a printer, an information processing device, a server, and a portable terminal.

The voltage-application control unit 105, the current response measurement unit 106, the target determination unit 108, the control unit (not shown) for controlling the overall operation of DPV detecting device 1, the display control unit (not shown) for the display unit, and the input/output control unit (not shown) for the data input/output unit and the input/output control unit (not shown) may be configured by dedicated circuitry, firmware, a computer program, hardware (such as a processor and a memory), and the like.

FIG. 3D shows a DPV detecting system including a DPV detecting device 1A and an information processing device 1B. DPV detecting device 1A includes a housing 101, an electrode chip connecting substrate 102, a display unit 103, an input operation unit 104, a voltage application control unit 105, a current response measurement unit 106, a data input/output unit 109, a radio communication unit (not shown), and a memory 1071. The memory 1071 stores measurement parameters, various control programs, and the like.

The information processing device 1B includes a data memory unit 107, a target determination unit 108, a communication unit (not shown), a processor (not shown), and the like. Elements with the same sign have the same function.

DPV detecting device 1A measures DPV at the electrode chip and calculates the current peak and the peak potential. The measured data including the calculated current peak and peak potential and the identification information of DPV detecting device is transmitted to the information processing device 1B. The target determination unit 108 of the information processing device 1B compares the received measured data with the peak-potential matching data stored in the data memory unit 107, and determines a target or a sign. The target determination unit 108 may also determine an estimated amount of the target.

The target determined by the target determination unit 108 may be sent to DPV detecting device 1A and displayed on the display unit 103. Further, it may be displayed by a monitor (not shown) of the information processing device 1B.

The information processing device 1B is not limited to a single DPV detecting device 1A, and can also receive measured data from a plurality of other DPV detecting device. A target is determined from each of the measurement data received from the plurality of devices. Various types of data such as measurement data and determination results are stored in the data memory unit 107.

[Image Analysis Device]

The image analysis device 2 illustrated in FIG. 4 is a device that acquires image data from the microscope 3 and determines a target. Examples of the microscope 3 include various microscopes such as a fluorescence microscope, a bright-field microscope, and a dark-field microscope.

The data acquisition unit 201 acquires image data observed by the microscope 3. The image data may be data captured by an image capturing unit of the microscope 3, or the data acquisition unit 201 may have a function of the image capturing unit. The microscope 3 and the data acquisition unit 201 may exchange data via a wired or wireless communication unit.

Imaging means include, for example, CCD cameras, CMOS cameras, color cameras and multi-spectrum cameras.

The image analysis unit 202 analyzes the color and/or shape of one or more specific bonding metal nanostructures from the image data of the binding body.

For example, the image analysis unit 202 specifies colors from RGB color data, color data, brightness data, spectral data, and the like by using various image-processing methods. The image analysis unit 202 calculates the area of the region of the specified color, and counts the number of regions of the color that are separated from each other.

The image analysis unit 202 uses various image processing techniques to identify the shape of the specific binding metal nanostructure and the shape of the binding body. The image analysis unit 202 calculates the area of the region of the specified shape, and counts the number of regions of the shape that are separated from each other.

The data memory unit 207 stores color shape matching data including at least color and/or shape specific to two or more specific binding metal nanostructures and data of two or more types of targets associated with the color and/or shape. The color shape matching data may be stored in advance in the data memory unit 207, or may be received and stored via the data acquisition unit 201 or a wireless communication unit (not shown), or may be updated at an appropriate timing.

In the present embodiment, the color shape matching data may include target identification information, target name, color information, shape information, target identification information, and target name.

The target determination unit 208 compares the color shape matching data with the color and/or the shape specified by the image analysis unit 202, and determines one or two or more types of targets.

In addition, the target determination unit 208 may calculate the number of cells or the number of virus particles from the area and the number of regions of the specified color and/or shape.

The display unit 4 displays the target determined by the target determination unit 208. Further, the display unit 4 may display the number of cells or the number of virus particles.

The microscope 3 and the display unit 4 may be provided in the image analysis device 2 or may be separate devices.

The display unit 4 is, for example, a liquid crystal monitor, an organic EL monitor, or the like.

The image analysis unit 202 may be configured by a processor, and may calculate a RGB of a color region from the color image data, and may extract a shape of the color region by distinguishing it from another. The target determination unit 208 may be configured by a processor, and may determine the specific binding metallic nanostructure and the type of the target by collating the collation data with the calculated RGB of the color region and/or the shape of the color region. As a result, two or more types of targets can be collectively detected.

[Wavelength Analysis Device]

The wavelength analysis device 6 is a device that acquires spectral data from the wavelength measuring means 5 and determines a target. Examples of the wavelength measuring means 5 include various wavelength measuring devices such as an absorbance measuring device, a fluorescence measuring device, and a scattered light measuring device.

The data acquisition unit 601 acquires spectral data measured by the wavelength measuring means 5.

The analysis unit 602 identifies a binding body in which one or more kinds of specific binding metal nanostructures (labels) and a target are bound from the spectral data.

The analysis unit 602 includes a processor, separates the waveforms of the spectral data obtained from two or more kinds of binding body into waveforms of the respective spectral data, and obtains the peak wavelength and the peak intensity from the waveforms of the respective spectral data.

The data memory unit 607 stores wavelength matching data including at least peak wavelength, peak intensity, and/or spectral data specific to two or more specific binding metal nanostructures and data of two or more types of targets associated with the peak wavelength, peak intensity, and/or spectral data. The wavelength matching data may be stored in advance in the data memory unit 607, or may be received and stored via the data acquisition unit 601 or a wireless communication unit (not shown), or may be updated at an appropriate timing.

In the present embodiment, the wavelength matching data may include target identification information, a target name, a wavelength, an intensity, spectral data, label identification information, and a label name.

The target determination unit 608 compares the wavelength matching data with the peak wavelength, the peak intensity, and/or the spectrum specified by the analysis unit 602, and determines one or more types of targets.

In addition, the target determination unit 608 may calculate an estimated amount of the target from the value of the peak intensity at the specified wavelength.

The display unit 4 displays the target determined by the target determination unit 608. Further, the display unit 4 may display an estimated amount of the target.

The wavelength measurement unit 5 and the display unit 4 may be provided in the wavelength analysis device 6 or may be separate devices.

The image analysis device 2 and the wavelength analysis device 6 may be configured by the information processing device or the computer program and hardware. The hardware includes, for example, a processor, a memory, a data bus, and the like.

[Detection Method: Electrochemical Detection]

The detection method includes a preparation step, an electrochemical detection step, a target determination step, and a determination result output step. DPV sensing device and the electrode chip described above may be used.

The preparation step may employ a plurality of different operating procedures (1), (2), (3) or (4).

The sample liquid is an aqueous solution containing the target.

The labeling liquid is a dispersion comprising at least two or more specific binding metal nanostructures.

The electrode is a single electrode consisting of a working electrode, a counter electrode, and a reference electrode.

(1) The sample liquid and the labeling liquid are mixed in advance, and a mixed solution containing a binding body in which the label is bound to the target is prepared. The mixture is then placed at least on the working electrode. After the mixture is placed on the electrode, the excess mixture may be removed with the washing liquid. Then, the measurement liquid is contacted on the electrode.

(2) The sample liquid and the labeling liquid are mixed in advance, and a mixture containing a binding body in which the label is bound to the target is prepared. The target to which the label is bound is then precipitated and centrifuged to separate the target to which the label is bound. The supernatant is discarded and an aqueous solution of water mixed with the separation is placed at least at the working electrode. Then, the measurement liquid is contacted on the electrode.

(3) The sample liquid is placed on the working electrode. It is to be noted that the excess mixture may be removed with the washing liquid after being laid. The labeling liquid is then contacted with the target in the sample liquid and the label is bound to the target. Then, the measurement liquid is contacted on the electrode.

(4) An electrode chip is used in which a plurality of labels is previously attached on or in the vicinity of the electrodes. The sample liquid is placed on the working electrode. After that, the excess mixture may be removed with the washing liquid. The measurement liquid is then injected over all the electrodes. As a result, the marker is separated from the surface of the electrode chip by the measurement liquid and is bound to the target. Note that the label may be separated from the surface of the electrode chip and bound to the target by the sample liquid.

In the above (1) to (4), the working electrode may have a predetermined surface roughness in order to supplement a target in an analyte, and a binding substance that binds to the target may be immobilized.

The electrochemical detection step measures the current response as a function of a predetermined range of voltage applied between the working electrode and the counter electrode. The peak of the current is detected, and the potential at that time is set as the peak potential, and the target can be determined from the peak potential.

The means for electrochemical detection used in the electrochemical detection step include, for example, a method using electrochemical techniques such as differential pulse voltammetry, normal pulse voltammetry, linear sweep voltammetry, stripping voltammetry, cyclic voltammetry, potentiometry, amperometry, and the like, controlled by a device for measuring current, voltage, electrical resistance, impedance, and software installed on the device.

The electrochemical detection step may include the following steps.

(1) Differential pulse voltammetry (DPV) manners are used. A voltage is applied between the working electrode and the counter electrode so that the pulse amplitude is constant and the base potential is increased in a predetermined number of steps, and the current response is measured.

(2) A current peak is detected from the measured current value, and a potential at the peak is detected. Since the peak potentials of the two or more specific binding metal nanostructures are different from each other, one or more peak potentials associated with the specific binding metal nanostructures bound to the target can be detected.

The target determination step determines a target corresponding to the peak potential or current peak. In this step, a target corresponding to each of the one or more peak potentials can be determined, and a plurality of types of targets can be collectively detected by two or more labels exhibiting different peak potentials.

The target determination step identifies one or more targets by matching the peak potential matching data with the measured peak potential of the specific binding metal nanostructure.

The peak potential matching data includes at least the specific binding metal nanostructure, attribute data including the peak potential, and target data associated with the attribute data.

The target determination step may include the following estimated amount calculation step.

(1) Estimated amount of the respective targets (analytes) is determined from the measured current peaks (e.g., peak height, area of peak shape (integral value)). The estimated amount may be of the order of digits.

For example, the estimated amount of the target may be determined from a preset current response value per single cell or virus and a current peak. The corresponding data of the current peak and the estimated amount is set in advance.

(2) The estimated amount may be determined using a preset calibration curve.

The determination result output step outputs the detected result.

Examples of the output include display on a display unit, output to a printer, print, output to an external device, and storage on a storage medium.

[Detection Method: Optical Detection]

The detection method includes a preparation step, an optical detection step, a target determination step, and a determination result output step. The microscope, the wavelength measurement means, the image analysis device, the spectrum analysis device, the slide glass, the optical cell, and the like described above may be used.

The preparation step may employ a plurality of different procedures:

(1) The sample liquid and the labeling liquid (dispersion containing specific binding metal nanostructures) are mixed in advance, and the labeling is bound to the target. The mixture is then dropped onto a slide glass and covered with a cover glass or placed in an optical cell.

(2) A label comprising a specific binding metal nanostructure of at least two or more different optical properties, which is pre-attached to the vicinity of the observation portion of the slide glass or to the inside of the optical cell, and a sample liquid are mixed on the slide glass or in the optical cell to bind the label to the target to obtain a binding body.

The optical detection step optically detects the target by confirming the binding body with the optical detection means.

Examples of the optical detection means include various kinds of microscopes such as a fluorescence microscope, a bright-field microscope, and a dark-field microscope, and various kinds of spectrum measuring instruments such as an absorbance meter, a fluorometer, and a spectrometer.

The optical detection step and the target determination step may include the following steps.

(1) The optical detection means confirms the color, shape, and size of the target on the slide glass. The light spot of the shape of the cell can be confirmed by binding of a label different in color from the bacterial color to the outer periphery of the target (e.g., bacteria). Different colors or wavelength peaks of the fluorescence or scattered light of the metal nanoparticles of the specific binding metal nanostructures that specifically bind to the target are different, so that a plurality of types of targets can be detected simultaneously and simultaneously.

(2) Differences in color can be used to identify the type of bacteria or virus, and the number, shape, or size of the identified cells (bacilli, cocci, and spirals) or virus particles can be used to determine the number (cell number, virus particle number). From the quantity per unit area, the quantity on the slide glass can be calculated on the order of digits.

The preparation step, the optical detection step and the target determination step may include the following steps.

(A-1) A mixture of the sample liquid and the labeling liquid (suspension) is allowed to stand, and the target to which the labels are bound is precipitated.

(A-2) Measure various optical intensities, such as absorbance or fluorescence intensity, of the supernatant with less labeling than initially.

(A-3) By comparing with various spectra such as absorbance or fluorescence intensity of the initial labeling liquid, it is possible to confirm the type of bacteria and virus from the difference in wavelength, and to detect which label precipitated together with the target. For example, the wavelengths of the absorption or fluorescence peaks are different for each label, so that they can be distinguished, and the intensities at the peaks quantitatively enable simultaneous collective detection of multiple types of targets. In other words, the unbound label is detected.

(A-4) The amount of precipitated label is estimated from various spectra, such as absorption or fluorescence peaks. The amount of target can also be estimated.

The preparation step, the optical detection step and the target determination step may include the following steps.

(B-1) A mixture of the sample liquid and the labeling liquid (suspension) is allowed to stand, the target to which the label is bound is precipitated, centrifuged, and the target to which the label is bound is separated.

(B-2) The supernatant is discarded and various optical intensities, such as absorbance or fluorescence intensity, of an aqueous solution of water mixed with the separation are measured.

(B-3) By comparing with various optical intensities such as absorbance or fluorescence intensity of the initial labeling liquid, various optical intensities such as absorbance or fluorescence intensity based on the label bound to the target are detected. For example, the wavelengths of the absorption or fluorescence peaks are different for each label, so that they can be distinguished, and the intensities thereof can quantitatively enable simultaneous collective detection of a plurality of types of targets. That is, the bound label is detected.

(B-4) The amount of label is estimated from various optical intensities, such as absorption or fluorescence peaks. The amount of target can also be estimated.

The preparation step, the optical detection step and the target determination step may include the following steps.

(C-1) Target and label binding bodies on a slide glass observed with various microscopes are captured by imaging means.

(C-2) The captured image data is analyzed by an image processing unit to obtain a color, a shape, and a size.

(C-3) The target is determined from the color, shape, and size obtained by the analysis. One or more targets may be identified by matching the color shape matching data with the analyzed color and shape. The color shape matching data includes at least attribute data including a color and a shape of the specific binding metal nanostructure and target data associated with the attribute data.

(C-4) From the color, shape, and size obtained by analysis, the quantity (the number of cells and the number of virus particles) is determined. From the quantity per unit area, calculate the quantity on the slide glass by the order of digits.

The target determination step determines a target corresponding to the measured peaks or spectra of various wavelengths. In this step, a target corresponding to each of the peaks and spectra of one or more various wavelengths can be determined, and simultaneous collective detection of a plurality of types of targets can be performed by two or more labels indicating peaks and spectra of different wavelengths.

The target determination step identifies one or more targets by matching peaks and spectral matching data of various wavelengths with peaks and spectra of various wavelengths of the measured specific binding metal nanostructures.

The peak and spectrum matching data of various wavelengths includes at least the specific binding metal nanostructure, attribute data including the peak and spectrum of the various wavelengths, and target data associated with the attribute data.

The target determination step may include the following estimated amount calculation step.

(1) Estimates of the respective targets (analytes) are determined from the measured wavelength peaks, spectra (e.g., maxima, area (integral)). The estimated amount may be of the order of digits.

As the estimated amount, for example, the amount of the target (the number of cells and the number of virus particles) may be determined from a predetermined wavelength peak, spectral intensity, measured wavelength peak, and spectral intensity per single cell (per unit area).

(2) The estimated amount may be determined using a preset calibration curve.

The determination result output step outputs the detected result.

Examples of the output include display on a display unit, output to a printer, print, output to an external device, and storage on a storage medium.

### Embodiment 1

Embodiment 1 shows an example of multi-item simultaneous detection of a plurality of types of bacteria or viruses by electrochemical measurement.

A sample liquid containing Salmonella, Escherichia coli O26, and Staphylococcus aureus is prepared as the target (sample).

The medium of the sample liquid is, for example, pure water. The sample liquid is adjusted to pH 6 to 7 with buffer liquid. For example, in the inspection of food poisoning, a pretreatment for separating and extracting bacteria adhering to a food material may be performed.

As a label, an iron-oxide particle (Fe₂O₃) modified with anti-Salmonella antibodies, a AuNP/PANI modified with anti-E. coli O26 antibodies, and a AgNP (silver nanoparticle) modified with anti-S. aureus antibodies were prepared.

(Preparation of AuNP/PANI)

Raspberry-like gold nanostructures consisting of gold nanoparticles (AuNP) and polyaniline (PANI) were prepared by redox reactions in aqueous solution.

(1) An aqueous solution of aniline (0.10 M, 10 mL) was added to an aqueous solution of chloroauric add (HAuCl₄) (0.0030 wt %, 500 mL) with vigorous stirring at 353 K for 20 minutes.

(2) The resulting dispersion was centrifuged at 8500 rpm, 278 K for 30 minutes. The supernatant was removed and the precipitate is dispersed in 50-mL ultrapure water. This procedure was repeated three times to remove unreacted species.

(3) The final precipitate was dispersed in 50-mL ultrapure water and stored at room temperature until use.

(4) Raspberry-type aggregates in which gold nanoparticles were dispersed in polyaniline are produced.

(Modification of AuNP/PANI Antibodies)

(1) A gold nanostructure dispersion (0.012 wt %, 25 mL) was mixed with a 2.0-mL 25% glutaraldehyde (GA) solution for 2 hours.

(2) The mixture was centrifuged at 8500 rpm and 278 K for 30 minutes. The supernatant was removed and the precipitate was resuspended in 25 mL ultrapure water. This procedure was repeated three times to remove excess unreacted GA.

(3) Anti-E. coli O26 antibodies (1.0 mg) were added with stirring the suspension for 2 hours.

(4) The resulting dispersion was centrifuged at 8500 rpm and 278 K for 30 minutes. The supernatant was removed and the pellets were resuspended in 15-mL ultrapure water and stored in the dark at 278 K until used.

In the presence of glutaraldehyde (GA), the amino group (polyaniline) and the amino group (antibody) are linked.

(Confirmation of Specific Binding to E. coli O26)

Dispersions of the antibody-introduced AuNP/PANI (50 μL) were added to a suspension of E. coli O26 (1.2×10⁸ CFU/mL, 450 μL), stirred at 298 K for 15 minutes, and 2 μL of the suspension was dropped onto a Si wafer, followed by electron-microscopy (SEM) observation.

(Preparation of Silver Nanoparticles (AgNP))

Disodium salt of EDTA (ethylenediaminetetraacetic add) (12 mg) as protective agents, and silver nitrate (0.1 M, 0.50 mL) were added to ultrapure water (0.10 L), and heated to 373 K (100° C.). Sodium hydroxide (1.0 M, 0.736 mL) was then added and the mixture was stirred for 3 minutes. After the solution turned yellow, it was stirred at room temperature for 2-3 hours. The silver nanoparticle dispersion was stored at room temperature.

EDTA adhered around the silver nanoparticles (AgNP) and formed a layer.

(Modification of AgNP Antibodies)

Since the AgNP dispersion had a pH of 11 or higher, the pH was adjusted with hydrochloric acid (about 1.0 M, 0.5 mL) to pH 7.0-7.5. Thiomalic acid (20 mM, 0.20 mL) was added to the pH-adjusted AgNP dispersion (9.8 mL) and the mixture was stirred at 298 K for 2 hours. Then, a triazine-based condensing agent (DMT-MM) (1.0 mg) was added to the dispersion, and the mixture was stirred at 278 K for 3 hours. After centrifuging the dispersion (8500 rpm, 278 K, 30 min), the supernatant was discarded, 10 mL of freshly ultrapure water was added and sonicated (100 Hz, 3 min). The dispersion was centrifuged again under the above conditions. The supernatant was discarded and 10 mL of new ultrapure water was added to obtain a dispersion. After that, 0.1 mg of antibodies (anti-O157 antibodies, or anti-S. aureus antibodies) were added and stirred at 298 K for 3 hours. The dispersion was stored in a refrigerator.

EDTA attached to the periphery of AgNP is removed, and thiomalic acid binds to the periphery of AgNP. The carboxyl group of thiomalic add binds to the amino group of the antibody. In place of the triazine-based condensing agent (DMT-MM), a carbodiimide-based condensing agent (EDC(1-Ethyl-3-(3-dimethylaminopropyl)carbodiimide)) may be used.

(Confirmation of Specific Binding to S. aureus)

The AgNPs introduced with the anti-Staphylococcus aureus antibody showed specific binding to S. aureus cells and no non-specific adsorption of the label to other than the target bacterium.

(Preparation of Iron-Oxide Nanoparticles (Fe₂O₃NP))

Iron chloride (3) hexahydrate (FeCl₃·6H₂O) (4.33 g) and iron (II) chloride tetrahydrate (FeCl₂·4H₂O) (1.57 g) were added and dissolved in pure water (12.5 mL) to which concentrated hydrochloric acid (12 M, 0.43 mL) had been added. The solutions are added dropwise to aqueous sodium hydroxide solution (NaOH) (1.5 M, 125 mL) with vigorous stirring. A black precipitate was collected with a magnet and the supernatant was removed. After washing with pure water three times, hydrochloric add (0.01 M, 250 mL) was added. The precipitate was separated by a magnet, washed twice with pure water, and then dispersed in an aqueous nitric acid solution (0.01 M). Fe₂O₃ was completely oxidized by stirring at 368 K for 1 hour. After returning to room temperature, the precipitate was washed twice with pure water and stored in a refrigerator.

(Modification of Fe₂O₃NP Antibodies)

Thiomalic acid (20 mM, 0.20 mL) was added to a dispersion of Fe₂O₃ NP (0.19 wt %, 9.8 mL) and stirred at 298 K for 2 hours. Then, a triazine-based condensing agent (DMT-MM)(1.0 mg) was added to the dispersion, and the mixture was stirred at 298 K for 3 hours. After centrifuging the dispersion (8500 rpm, 278 K, 30 min), the supernatant was discarded, 10 mL of ultrapure water was newly added, and sonicated (28 Hz, 3 min). The dispersion was centrifuged again under the above conditions. The supernatant was discarded and a new 10 mL of ultrapure water was newly added to obtain a dispersion. Then, anti-Salmonella antibody (1 mg/mL, 10 μL) is added to the dispersion (1 mL), and the mixture was stirred for 24 hours. The dispersion was stored in a refrigerator.

(Confirmation of Specific Binding to Salmonella)

The antibody-introduced Fe₂O₃NP dispersion (50 μL) was added to a suspension of Salmonella (3.9×10⁸ CFU/mL, 450 μL) and the mixture was stirred at 298 K for 15 minutes, and after dropping 2 μL of the mixture onto a Si wafer, observation was made with an electron-microscopy (SEM).

The prepared three types of labels (metal nanoparticle structures) were mixed with pure water to prepare a label dispersion.

<Examples of Other Labels>

1. Target: Escherichia coli O157, Label: AgNP

(Preparation of AgNP)

Disodium salt of EDTA (ethylenediaminetetraacetic acid) (12 mg) as a protective agent, and silver nitrate (0.1 M, 0.50 mL) were added to ultrapure water (0.10 L) and heated to 373 K (100° C.). Sodium hydroxide (1.0 M, 0.736 mL) was then added and the mixture was stirred for 3 minutes. After the solution turned yellow, it was stirred at room temperature for 2-3 hours. AgNP dispersions were stored at room temperature.

EDTA adhered around AgNP to form a layer.

(Modification of AgNP Antibodies)

Anti-O157 antibodies were added together with a catalyst to the dispersion of AgNP with carboxy-group modification on the surface, and the mixture was stirred at 278 K for 24 hours to introduce the antibodies on the surface of AgNP.

(Confirmation of Specific Binding to E. coli O157)

The dispersions of the antibody-introduced AgNPs each were added to a suspension of E. coli O157 and E. coli O26 (10⁷ cells/mL), and each mixture was stirred at room temperature, and after 30 minutes, 2 μL of each mixture was dropped onto a Si wafer, and observation was executed with an electron-microscopy (SEM). In SEM image, insulating E. coli was observed as a black rod-like contrast and the conductive AgNP as white aggregates. It was observed that AgNP bind to all E. coli O157 cells but not with E. coli O26 cells at all. From these findings, it was confirmed that the antibody-modified AgNP specifically bind to O157 cells. In addition, it was confirmed that there is no non-specific adsorption of the label to other than the target bacteria.

(Preparation of PdNP)

(1) A palladium-chloride solution (56 mM, 3 mL) was added to ultrapure water (0.1 L) and heated to 353K.

(2) An aqueous solution (5.6 mL) containing trisodium citrate (24 mM) and sodium ascorbate (29 mM) was added to the solution with stirring at 353 K for 30 minutes.

(Preparation of PdNP/PANI)

Preparation of Raspberry-Like Metal Nanostructures Consisting of Palladium Nanoparticles (PdNP) and Polyaniline (PANI).

(1) A palladium chloride solution (1 wt %, 0.131 mL) was added to ultrapure water (0.1 L) and heated to 353 K.

(2) An aniline solution (0.1 M, 20 mL) was added to the solution with vigorous stirring at 353 K for 20 minutes.

(3) The resulting dispersion were centrifuged at 8500 rpm, 278 K for 30 minutes. The supernatant was removed and the precipitate was dispersed in 40-mL ultrapure water. This procedure was repeated three times to remove unreacted species.

(4) The final precipitate was dispersed in 10 mL ultrapure water and stored at room temperature until use.

(5) Raspberry-like PdNP/PANI aggregates in which palladium nanoparticles are dispersed in polyaniline were produced.

(Preparation of Copper Oxide Nanoparticles (Cu₂ONP))

(1) A copper sulfate solution (0.1 M, 1.6 mL) and a hexadecyltrimethylammonium chloride solution (0.32 M, 5 mL) were added to ultrapure water (0.1 L), and nitrogen bubbling was performed in a sealed condition for 30 minutes.

(2) Adding a sodium borohydride solution (1.1 M, 2.5 mL), then the mixture was stirred at 298 K for 12 h with nitrogen bubbling.

(3) The resulting solutions were centrifuged at 1300 rpm, 278 K for 30 minutes. The supernatant was discarded and the precipitate was dispersed in 100-mL ultrapure water. This procedure was repeated three times to remove unreacted species.

(4) The final precipitate was dispersed in 100-mL ultrapure water and stored in a refrigerator until use.

(Preparation of Tin Nanoparticles (SnNP))

(1) Tin chloride dihydrate (0.5 g) and polyvinylpyrrolidone K30 (0.15 g) were dissolved in an ethylene glycol solution (0.1 L).

(2) Sodium borohydride (0.5 g) was added, and the mixture was stirred at 298 K for 30 minutes.

(3) Acetone (100 mL) was added, and the mixture was centrifuged at 13000 rpm, 298 K for 30 minutes. The supernatant was removed and the precipitate was dispersed in 100-mL ultrapure water. This procedure was repeated three times to remove unreacted species.

(4) The final precipitate was dried in vacuo with 343 K.

(5) The resulting powder is dispersed in 50-mL ultrapure water.

(Preparation of AuNP/PmPD)

A Gold nanostructure was prepared by a redox reaction in aqueous solution.

(1) m-phenylenediamine (54.1 mg) was added and dissolved in ethanol (5 mL).

(2) The m-phenylenediamine solution (0.1 M, 4 mL) was added to an aqueous solution of HAuCl₄ (0.0030 wt %, 200 mL) with vigorous stirring at 353 K for 20 minutes.

(3) The resulting dispersion was centrifuged at 8500 rpm, 278 K for 30 minutes. The supernatant was removed and the precipitate was dispersed in 50 mL ultrapure water. This procedure was repeated three times to remove unreacted species.

(4) The final precipitate was dispersed in 20-mL ultrapure water and stored at room temperature until use.

(5) Raspberry-type aggregates in which gold nanoparticles are dispersed in poly-m-phenylenediamine were produced.

(Preparation of AuNP/PmAP, or PoAP)

A gold nanostructure was prepared by a redox reaction in aqueous solution.

(1) m-aminophenol or o-aminophenol (54.6 mg) was added and dissolved in ethanol (5 mL).

(2) A solution of m-aminophenol or o-aminophenol (0.1 M, 4 mL) was added to an aqueous solution of HAuCl₄ (0.0030 wt %, 200 mL) with vigorous stirring at 353 K for 20 minutes.

(3) The resulting dispersion was centrifuged at 8500 rpm, 278 K for 30 minutes. The supernatant was removed and the precipitate is dispersed in 50-mL ultrapure water. This procedure is repeated three times to remove unreacted species.

(4) The final precipitate was dispersed in 20-mL ultrapure water and stored at room temperature until use.

(5) A raspberry-type aggregate in which gold nanoparticles were dispersed in a poly-m-aminophenol or a poly-o-aminophenol was produced.

(Preparation of AuNP/PmAB, or PoAB)

A gold nanostructure was prepared by a redox reaction in aqueous solution.

(1) m-aminobenzoic acid or o-aminobenzoic add (68.8 mg) was added and dissolved in ethanol (5 mL).

(2) The m-aminobenzoic add or o-aminobenzoic acid (0.1 M, 4 mL) was added to an aqueous solution of HAuCl₄ (0.0030 wt %, 200 mL) with vigorous stirring at 353 K for 20 minutes.

(3) The resulting dispersion was centrifuged at 8500 rpm, 278 K for 30 minutes. The supernatant was discarded and the precipitate was dispersed in 50-mL ultrapure water. This procedure was repeated three times to remove unreacted species.

(4) The final precipitate was dispersed in 20-mL ultrapure water and stored at room temperature until use.

(5) A raspberry-type aggregate in which gold nanoparticles are dispersed in poly-m-aminobenzoic add or o-poly-aminobenzoic acid was produced.

(Preparation of AuNP/PmTD)

(1) m-toluidine (53.6 μL) was added and dissolved in ethanol (5 mL).

(2) The m-toluidine solution (0.1 M, 4 mL) was added to an aqueous solution of HAuCl₄ (0.0030 wt %, 200 mL) with vigorous stirring at 353 K for 20 minutes.

(3) The resulting dispersion was centrifuged at 8500 rpm, 278 K for 30 minutes. The supernatant was discarded and the precipitate was dispersed in 50-mL ultrapure water. This procedure was repeated three times to remove unreacted species.

(4) The final precipitate was dispersed in 20-mL ultrapure water and stored at room temperature until use.

(5) A raspberry-type aggregate in which gold nanoparticles are dispersed in a poly-m-toluidine was produced.

Photographs of the labels taken by electron-microscopy are shown in FIGS. 12A and 12B.

<Metal Nanostructures>

Zeta potential and particle size analyzer (ELSZ-2plus, Otsuka Electronics Co., Ltd.) particle size and zeta potential were measured by dynamic light scattering method. The arithmetic mean diameter over count was taken.

TABLE 1

| | Diameter of Label particle | Zeta |
|---|---|---|
| Label | (mean diameter) | potential |
| particle | nm | mV |
| AuNP/PANI | 90.4 | −25.55 |
| PdNP/PANI | 25.2 | −14.38 |
| AgNP/PANI | 59.8 | −20.4 |
| CuNP/PANI | 105.7 | −4.91 |
| AuNP/PmPD | 130.4 | −11.98 |
| AuNP/PmAP | 154 | −16.89 |
| AuNP/PoAP | 82 | −14.32 |
| AuNP/PoAB | 99.9 | −27.03 |
| AuNP/PmAB | 102.7 | −32.57 |
| AuNP/PmTD | 128.6 | −6.5 |
| AuNP small | 5.5 | −10.78 |
| AuNP medium | 28.9 | −32.5 |
| AuNP large | 81.9 | −56.76 |
| AgNP small | 2.8 | −35.7 |
| AgNP medium | 30.1 | −19.67 |
| AgNP large | 91 | −37.86 |
| Fe₂O₃ | 66.2 | −28 |
| Cu₂ONP | 99.6 | −33.23 |
| SnNP | 83.2 | −32 |
| PdNP | 88.7 | −28.32 |
| ZnONP | 151.2 | −34.61 |
| CdSe/ZnSNP | 2.8 | — |
| CdSe/ZnSNP | 4.7 | — |

In the label particle, adopting a raspberry-like aggregate or an aggregate of nanoparticles, of which the surface area is larger than that of a simple nanoparticle, enables to increase the current response.

In addition, in a raspberry-like aggregate having a structure in which small metal nanoparticles are covered with a polymer, their current response can be controlled by the polymer and the metal nanoparticles. For example, in a raspberry-like aggregate with small current response AuNP, a current response of a conductive polymer can be used. In a raspberry-like aggregate with large current response metal nanoparticles, by which the current response of the conductive polymer is observed to be small, the current response of the metal nanoparticles can be used. A large number of metal nanoparticles constituting the raspberry-like aggregate have a small particle size with a large surface area, and thus the aggregate functions as a highly sensitive label with large current response even in the case that its metal content is smaller than simple nanoparticles.

<Modification of Metal Nanostructures to Antibodies>

The results of antibody modification by the label particles are shown in Table 2.

TABLE 2

| | Antibody species | | | | | | |
|---|---|---|---|---|---|---|---|
| Label | | | | | S. | Noro | Influenza |
| particle | Enterobacteriaceae | O157 | O26 | Salmonella | aureus | virus | virus |
| AuNP/PANI | ∘ | ∘ | ∘ | ∘ | ∘ | | |
| PdNP/PANI | | | | | | ∘ | ∘ |
| CuNP/PANI | ∘ | | ∘ | ∘ | ∘ | | |
| AuNP/PmPD | ∘ | | ∘ | ∘ | ∘ | | |
| AuNP/PmAP | ∘ | | ∘ | ∘ | ∘ | | |
| AuNP/PoAP | ∘ | | ∘ | ∘ | ∘ | | |
| AuNP/PoAB | ∘ | | ∘ | ∘ | ∘ | | |
| AuNP/PmAB | ∘ | | ∘ | ∘ | ∘ | | |
| AuNP/PmTD | ∘ | | ∘ | ∘ | ∘ | | |
| AuNP small | | | | | | ∘ | ∘ |
| AuNP medium | ∘ | | ∘ | ∘ | ∘ | | |
| AgNP small | | | | | | ∘ | ∘ |
| AgNP medium | ∘ | ∘ | ∘ | ∘ | ∘ | | |
| Fe₂O₃ | ∘ | | ∘ | ∘ | ∘ | | |
| Cu₂ONP | ∘ | | ∘ | ∘ | ∘ | | |
| SnNP | ∘ | | ∘ | ∘ | ∘ | | |
| PdNP | ∘ | | ∘ | ∘ | ∘ | | |
| ZnONP | ∘ | | ∘ | ∘ | ∘ | | |

(Setting the Current Response Per Single Cell)

For each label, the current response per single cell or virus particle (per unit area) for was set. The dispersion containing the label was dropped onto a carbon disk electrode and dried, and electrochemical measurement was performed to confirm current responsiveness.

(1) It was observed that AuNP/PANI (100 nm) had a current response of 950 nA (−0.13 V), and a label that is capable of detecting a single cell (geometric surface area: 3.8×10⁻⁷ cm²) with a current response of 1.0 nA was prepared.

(2) It was observed that AgNP (30 nm) had a current response of 22 μA (+0.11 V), and by using the AgNP, a label that is capable of detecting a single cell (geometric surface area: 3.8×10⁻⁷ cm²) with a current response of 24 nA was prepared. It was observed that AgNP/PANI (100 nm) has a peak potential at +0.42V and a current response of 5.1 μA, and by using the AgNP/PANI, a label that is capable of detecting a single cell (geometric surface area: 3.8×10⁻⁷ cm²) with a current response of 55 nA was prepared.

(3) It was observed that Fe₂O₃NP has a current response of 1.6 μA (−0.25 V), and by using Fe₂O₃NP, a label that is capable of detecting a single cell (geometric surface area: 3.8×10⁻⁷ cm²) with a current response of 1.7 nA was prepared.

The current responses of the other target particles (peak potential, peak current value, and current density by DPV measurement) are shown in Table 3.

TABLE 3

| | Peak | | | |
|---|---|---|---|---|
| | potential | Peak | Current | single |
| Label | (vs. Ag \| AgCl) | current | density | cell |
| particle | (V) | (nA) | (mA/cm⁻²) | (nA/cell) |
| AuNP/PANI | −0.13 | 950 | 2.7 | 1 |
| PdNP/PANI | 0.31 | 7.5 | 0.021 | 0.0081 |
| AgNP/PANI | 0.42 | 5100 | 140 | 55 |
| CuNP/PANI | 0.084 | 4800 | 0.069 | 0.026 |
| AuNP/PmPD | −0.07 | 430 | 1.2 | 0.47 |
| AuNP/PmAP | −0.11 | 8.2 | 0.023 | 0.0089 |
| AuNP/PoAP | −0.22 | 8.2 | 0.023 | 0.0089 |
| AuNP/PoAB | −0.16 | 1000 | 2.8 | 1.1 |
| AuNP/PmAB | −0.33 | 430 | 1.2 | 0.46 |
| AuNP/PmTD | −0.11 | 7500 | 22 | 8.1 |
| AuNP small | 1.53 | 47100 | 130 | 51 |
| AuNP medium | 1.56 | 12900 | 36 | 14 |
| AuNP large | 1.43 | 4270 | 12 | 4.6 |
| AgNP small | 0.1 | 61800 | 170 | 66 |
| AgNP medium | 0.11 | 21900 | 62 | 24 |
| AgNP large | 0.1 | 15900 | 45 | 17 |
| Fe₂O₃ | −0.25 | 1600 | 4.6 | 1.7 |
| Cu₂ONP | −0.09 | 24000 | 69 | 26 |
| SnNP | 0.46 | 37 | 0.11 | 0.04 |
| PdNP | −0.08 | 4200 | 1.2 | 0.45 |
| ZnONP | −1.21 | 6655 | 19 | 7.2 |
| CdSe/ZnSNP | −0.92 | 140 | 0.4 | 0.15 |
| (2.8 nm) | | | | |

The following points can be seen from Tables 1 and 3.

Comparing the electrode, in which 70 ng of metal nanoparticles, AgNP with different particle size (2.8 nm, 30.1 nm, 91 nm) was attached, the small particles showed no less than three times larger current response than the large particles, respectively. Comparing the electrode, in which 70 ng of metal nanoparticles, AuNP with different particle size (5.5 nm, 30.1 nm, 91 nm) was attached, the small particles showed no less than 10 times larger current response than the large particles, respectively. In order to use these specific surface area effect, a composite (AgNP/PANI or AuNP/PANI) composed of small metal nanoparticles (AgNP or AuNP) was formed, and this composite enables to obtain a higher current response while saving the amount of metal to be used than that of metal nanoparticles of the same size.

From Table 3, it is evident that the current values of the label particles converted per single cell differ greatly. According to this result, the sensitivity (current value) in the measurement is capable of being adjusted by choosing a label particle (for example, small current response label particles are for bacteria/viruses contained in a sample liquid in a large number, or large current response label particles are for bacteria/viruses that is harmful to the human body even in a very small number).

### Example 1

(A1) By mixing a sample liquid containing Salmonella, E. coli O26, and S. aureus as targets (samples) with a labeled dispersion containing an anti-Salmonella antibody-modified Fe₂O₃, an anti-Escherichia coli O26 antibody-modified AuNP/PANI, and an anti-S. aureus antibody-modified AgNP, the corresponding antigens were bound to the antibodies by an antigen-antibody response.

(A2) A differential pulse voltammetry (DPV) detector (electrochemical analyzer Model 830D manufactured by ALS) with a single electrode consisting of a working electrode, a reference electrode, and a counter electrode was used to measure the current response of a target-label (binding body), and each different peak potential was measured, and a current value at each peak potential was displayed on a monitor. As a result, three types of targets were simultaneously detected at once. In addition, since the current response per single cell could be set, the number of cells can be determined.

The bacteria were as follows:
Salmonella (3.9×10⁹ cells/mL)
Escherichia coli O26 (1.7×10⁹ cells/mL)
Staphylococcus aureus (4×10⁸ cells/mL)

Labels are as follows:
Fe₂O₃-anti-Salmonella antibody (10 μg/mL)
AuNP/PANI-anti-O26 antibody (10 μg/mL)
AgNP-anti-S. aureus antibody (10 μg/mL)

The measurement procedure was as follows.

1. Stock dispersion of each bacterium were diluted 100 times with sterile water.

2. 50 μL of the diluted bacterial solutions and 50 μL of the labeled antibody solution in each bacteria are mixed and stirred at 25° C. for 15 minutes.

3. Each three mixed solutions containing bacteria and labeled antibody was dispensed in 50-μL portions, and mixed together, and stirred for 10 seconds. A mixed solution is obtained.

4. 5 μL of the mixed solution was dropped onto the working electrode of the measuring apparatus, then the electrode was allowed to stand for 5 minutes, and rinsed with pure water.

5. 30 μL of phosphate buffer solution (Phosphate buffer: PB) was dropped onto the electrode (single electrode consisting of working electrode, referencing electrode, and counter electrode), and DPV measurement was executed.

FIG. 6 shows current values at different peak potentials. FIG. 6 shows that Salmonella is detected at the first peak potential, E. coli O26 is detected at the second peak potential, and S. aureus is detected at the third peak potential.

Each peak value of the current response was obtained by determining a baseline from the current response curve and calculating a peak height.

### Example 2

The mixed solution (a mixture of the target and the label) obtained in the measurement procedure 3. of Example 1 was measured using optical detection means such as a fluorescence microscope, a dark-field microscope, and a spectrum, and three types of targets were simultaneously detected at once.

FIG. 7 is an image taken with a dark-field microscope. Three bacteria types could be detected by labeling with different colors (blue, white, orange). Conventional Gram-stained light microscopy, which uses agents that distinguish between two genera, Gram-negative and Gram-positive, cannot easily distinguish between both Gram-negative Salmonella and Escherichia coli O26, and therefore distinguishing these bacteria depends on the observers' experience. On the other hand, according to the present example, two types of gram-negative bacteria can be easily distinguished with a difference in color.

FIG. 8 shows the results detected by the scattered light spectrum measuring apparatus. FIG. 8 shows the spectrum from the labels bound to a single bacterium (one individual). Three types of bacteria were detected as different wavelength peaks. The numbers of the cells can also be estimated by their respective intensities. If the number of cells is multiple, multiple spectra may also be measured to estimate the number of cells from the total number of spectra.

The spectral waveform may be an actual measurement value or an approximate curve of the actual measurement value, depending on the measurement apparatus. A wavelength peak may be determined in the approximation curve.

In Examples 1 and 2, three labels (antibody-modified metal nanostructures) having non-overlapping current response (peak potential), color, and wavelength peak were selected, and a plurality of targets were accurately distinguished simultaneously by electrochemical test and optical inspection.

### Example 3

The bacteria are as follows:
Enterobacteriaceae (Salmonella, E. coli, and E. coli O26) (1.2×10⁹ cells/mL each)
S. aureus (4×10⁸ cells/mL)

Labels are as follows:
Fe₂O₃-anti-Enterobacteriaceae antibodies (10 μg/mL)
AgNP-anti-S. aureus antibody (10 μg/mL)

The measurement procedure is as follows.

1. Stock dispersions of each bacterium are diluted 100 times with sterile water.

2. 50 μL of the diluted bacterial solutions and 50 μL of the labeled antibody solution in each bacterium are mixed and stirred at 25° C. for 15 minutes.

3. The dispersions of two types of the bacteria and the solution of labeled antibody were taken 50 μL each, and mixed with shake for 10 seconds to obtain a mixed solution.

4. 5 μL of the mixed solution was dropped onto the working electrode of the measuring apparatus, then the electrode was allowed to stand for 5 minutes, and rinsed with pure water.

5. 30 μL of phosphate buffer solution (PB) is dropped onto the electrode (single electrode consisting of working electrode, reference electrode, and counter electrode), and DPV measurement was executed (using an electrochemical analyzer Model 830D manufactured by ALS).

FIG. 9A shows the current values at different peak potentials. FIG. 9A shows that the Enterobacteriaceae group was detected at the first peak potential and S. aureus was detected at the second peak potential. A similar experiment was conducted using AuNP/PANI-anti-Enterobacteriaceae antibody (10 μg/mL) instead of Fe₂O₃-anti-Enterobacteriaceae antibody, and the results are shown in FIG. 9B. FIG. 9B shows that the Enterobacteriaceae was detected at the first peak potential and S. aureus was detected at the second peak potential.

In FIGS. 9A and 9B, the same AgNP-anti-S. aureus antibody was used, but the position of the second peak was slightly shifted and the intensity differed by orders of magnitude. However, the difference was within acceptable limits as a measurement error and detection accuracy of the measurement in terms of the relationship with other labels, the degree of binding to the target.

### Example 4

The mixed solution (a mixture of a target and a label) obtained in the measurement procedure 3 of Example 3 was measured using an optical detection means such as a fluorescence microscope, a dark-field microscope, or an absorption spectrometer, and two types of targets were simultaneously detected at once.

FIG. 10A is a dark-field microscopy image with Fe₂O₃-anti-Enterobacteriaceae antibodies and AgNP-anti-S. aureus antibodies. FIG. 10B is a dark-field microscopy image using AuNP/PANI-anti-Enterobacteriaceae group antibodies and AgNP-anti-S. aureus antibodies. It was confirmed that one bacterial group and one bacteria specie were detected with differently colored labelling.

FIGS. 11A and 11B were the results from the scattered-light spectrometer. The spectra of labels bound to each bacterium (one individual) were shown. Different wavelength peaks distinguished S. aureus from Enterobacteriaceae. In addition, three species of the Enterobacteriaceae bacteria could be detected with different intensities but similar wavelength peaks. These species had a slightly different spectral intensity because of the different sizes of the bacteria (number of antigens).

The number of the bacteria cells can also be estimated from the area of each intensity or peak position of the spectrum. If the number of cells is multiple, multiple spectra may also be measured, and the number of cells may be estimated from the total number of spectra.

### Example 5

In Example 5, both viruses and bacteria were detected simultaneously at a single electrode.

A single-electrode DPV detector (electrochemical analyzer Model 830D manufactured by ALS) consisting of a working electrode, a reference electrode, and a counter electrode was used to measure the current response.

(1) Target (sample)
Escherichia coli O26: 1.1×10⁷ cells/mL
Influenza virus: 1000 ng/mL (6.0×10⁷ particles/mL)

(2) Antibody-modified metal nanostructures (label-antibody)
AuNP/PmTD-anti-O26 antibody-AgNP-anti-influenza antibody

(3) Experimental procedure
i) 20 μL of anti-influenza antibody-modified AgNP and 10 μL of 1000 ng/mL (6.0×10⁷ particles/mL) influenza virus (6.0×10⁵ particles) were mixed and stirred for 15 minutes.
ii) 20 μL of anti-E. coli O26 antibody-modified AuNP/PmTD and 10 μL of O26 suspension (1.1×10⁷ cells/mL) (1.1×10⁵ cells) were mixed and stirred for 15 minutes.
iii) 10 μL of each suspension was taken and mixed to obtain a sample.
iv) 3 μL of the sample was dropped onto the working electrode of the measuring apparatus to stand for 5 minutes, and pure water was poured.
v) 30 μL of phosphate buffer liquid (PB) was dropped onto the electrode (single electrode consisting of working electrode, referencing electrode, and counter electrode), and then DPV was measured.

In FIG. 15, the response of AuNP/PmTD-anti-influenza antibody was found at the first peak potential, and the response of AgNP-anti-influenza antibody was found at the second peak potential.

### Example 6

In Example 6, two virus species are simultaneously detected at a single electrode.

A DPV detector (electrochemical analyzer Model 830D manufactured by ALS) having a single electrode composed of a working electrode, a reference electrode, and a counter electrode was used to measure the current response.

(1) Target (Sample)
Norovirus: 1 ng/mL (5.1×10⁷ particles/mL)
Influenza virus: 1000 ng/mL (6.0×10⁷ particles/mL)

(2) Antibody-Modified Metal Nanostructures (Label-Antibody)
AgNP-Anti-norovirus antibody (Anti-Caliciviridae (1B1))
AuNP-anti-influenza antibody

(3) Experimental Procedure
i) 20 μL of the anti-norovirus antibody-modified AgNP and 10 μL of 1 ng/mL (5.1×10⁷ particles/mL) norovirus (5.1×10⁵ particles) were mixed and stirred for 15 minutes.
ii) 20 μL of anti-influenza antibody-modified AuNP and 10 μL of 1000 ng/mL (6.0×10⁷ particles/mL) influenza virus (6.0×10⁵ particles) were mixed and stirred for 15 minutes.
iii) 10 μL of each solution was taken and mixed to obtain a sample.
iv) 3 μL of the sample was dropped onto the working electrode of the measuring apparatus to stand for 5 minutes, and pure water was poured.
v) 30 μL of phosphate buffer solution (PB) was dropped onto the electrode (single electrode composed of working electrode, referencing electrode, and counter electrode), and DPV was measured.

In FIG. 16, the response of AgNP-anti-norovirus antibody was found at the first peak potential, and the response of AuNP-anti-influenza antibody was found at the second peak potential.

### Example 7

The influenza virus and E. coli O26 were simultaneously detected by optical inspection.

(1) Target (Sample)
Escherichia coli O26: 1.1×10⁷ cells/mL
Influenza virus: 1000 ng/mL (6.0×10⁷ particles/mL)

(2) Antibody-Modified Metal Nanostructures (Label-Antibody)
AuNP (Large)-Anti O26
AgNP-anti-influenza antibodies

(3) Experimental Procedure
i) 20 μL of anti-influenza antibody-modified AgNP and 10 μL of 1000 ng/mL (6.0×10⁷ particles/mL) influenza virus (6.0×10⁵ particles) were mixed and stirred for 15 minutes.
ii) 20 μL of an anti-E. coli O26 antibody-modified AuNP (large) was mixed with 10 μL of a 1.1×10⁷ cells/mL O26 suspension (1.1×10⁵ cells) and stirred for 15 minutes.
iii) 10 μL of each solution was taken and mixed to obtain a sample.
iv) 10 μL of the sample was dropped onto a glass slide, dried, and then observed with a dark-field microscope.

FIG. 17 is a dark field microscopy image obtained by using a AuNP (large)-anti-O26 antibody and a AgNP-anti-influenza antibody. It was confirmed that Escherichia coli O26 and influenza virus were detected with differently colored labeling.

FIG. 18 shows the results detected by the scattered light spectrum measuring apparatus. The spectra of labels bound to a bacterium (one individual) and a single virus particle was shown. In FIG. 18A, a large wavelength peak is a wavelength of the binding body of E. coli O26 and AuNP (large)-anti O26 antibody, and a small wavelength peak is a wavelength of E. coli O26. In FIG. 18B, a large wavelength peak is the wavelength of the binding body of the influenza virus and AgNP-anti-influenza antibody, and a small wavelength peak is the wavelength of the influenza virus. It is difficult to distinguish E. coli O26 and influenza viruses that did not bind a label from other types of bacteria and viruses because they are appears as peaks in the same wavelength range. On the other hand, it is possible to reliably distinguish and detect different targets by measuring the wavelength peak in the present invention, since a target and a label are bound as a binding body and the peak wavelength unique to each label can be set in advance.

### Example 8

An example using a single-electrode DPV detector (eBacSens) consisting of a working electrode, a reference electrode, and a counter electrode, and a single-electrode DPV detector consisting of a working electrode, a reference electrode, and a counter electrode (electrochemical analyzer Model 830D manufactured by ALS) in FIGS. 1A and 3A is shown.

(1) Concentration of the Bacterial Stock Liquid
S. aureus: 4.8×10⁸ cells/mL
E. coli O26: 1.1×10⁹ cells/mL
E. coli (NBRC3972): 1.1×10⁹ cells/mL
Salmonella: 1.1×10⁹ cells/mL

(2) Antibody-Modified Metal Nanostructures (Label-Antibody)
Fe₂O₃-anti-Enterobacteriaceae (ECA) antibody
AgNP-anti-S. aureus antibody

(3) Experimental Procedure

The measurement procedure was performed as described in Example 3 above.

The labels were not attached to the electrode chip in advance.

(4) The results of the peak potential and the peak current value are shown in Table 4.

TABLE 4

| | | Peak potential | Peak current |
|---|---|---|---|
| | Label | V vs Ag \| AgCl | μA |
| DPV detector | Fe₂O₃-anti- | −0.325 | 3.21 |
| | Enterobacteriaceae | | |
| | (ECA) antibody | | |
| | AgNP-anti-S. | 0.015 | 2.15 |
| | aureus antibody | | |
| eBacSens | Fe₂O₃-anti- | −0.415 | 3.15 |
| | Enterobacteriaceae | | |
| | (ECA) antibody | | |
| | AgNP-anti-S. | 0.05 | 2.41 |
| | aureus antibody | | |

(5) FIG. 13A is the measurement result displayed on the DPV detector (eBacSens). Display 103 displayed that two types of labels were detected. Two kinds of labels could be detected because the positions (potentials) of the peak of the determined current response were different.
(1) Name: Fe₂O3
Peak: 3.15 μA
BG: 3.37 μA
(2) Name: AgNP
Peak: 2.41 μA
BG: 2.58 μA

(6) FIG. 13B is the data of the current response curve on the computer screen connected to it and the output results of two different peak potentials.

FIG. 14 is the current response curve data measured by DPV detector and the output results of the two types of peak potentials.

In both apparatus, two types of peak potentials could be detected, and thus two types of targets could be accurately identified.

### Example 9

Here is an example using a DPV detector (eBacSens) of single electrode containing a working electrode, a reference electrode, and a counter electrode, and a DPV detector of single electrode containing a working electrode, a reference electrode, and a counter electrode (electrochemical analyzer Model 830D manufactured by ALS) shown in FIGS. 1A and 3A.

(1) Concentration of the Bacterial Stock Liquid

(Condition A)
E. coli O26: 3.2×10⁹ cells/mL
Salmonella: 5.0×10⁹ cells/mL

(Condition B)
E. coli O26: 6.5×10⁷ cells/mL
S. aureus: 5.0×10⁶ cells/mL

(Condition C)
E. coli O26: 6.5×10⁷ cells/mL
E. coli O157: 5.0×10⁶ cells/mL

(2) Antibody-Modified Metal Nanostructures (Label-Antibody)

(Condition A)
AuNP/PmTD-anti-O26 antibody (10 μg/mL)
CuNP/PANI-anti-Salmonella antibody (10 μg/mL)

(Condition B)
AuNP/PmTD-anti O26 antibody (4 μg/mL)
CuNP/PANI-anti-S. aureus antibody (7 μg/mL)

(Condition C)
AuNP/PmTD-anti-O26 antibody (4 μg/mL)
CuNP/PANI-anti O157 antibody (7 μg/mL)

(3) Experimental Procedure

The measurement procedure was as follows.

1. 50 μL of each bacterial solution under each condition and 50 μL of the labeled antibody solution corresponding to the bacteria are mixed and stirred at 25° C. for 15 minutes to obtain a mixed solution containing either of the bacteria and its labeled antibody.

2.1 (Condition A: E. coli O26 and Salmonella)
1.0 μL of a mixed solution containing E. coli O26 and the labeled antibody for E. coli O26 was added to 0 μL of a mixed solution containing Salmonella and the labeled antibody for this bacteria, then mixed. The mixed solution was dropped onto the working electrode of the measuring apparatus to stand for 5 minutes, and ultrapure water was poured. Then, DPV measurement of Step 3 was executed.
In the above, the same procedure was executed by changing the amount of the mixed solution containing Salmonella and labeled antibody for this bacteria to 0.2 μL, 0.5 μL, 1.0 μL, or 2.5 μL.

2.2 (Condition B: E. coli O26 and S. aureus)
1. 53 μL of a mixed solution containing E. coli O26 and the labeled antibody for E. coli O26 was added to 0 μL of a mixed solution containing S. aureus and the labeled antibody for S. aureus, then mixed, and the mixed solution was dropped onto the working electrode of the measuring apparatus to stand for 5 minutes, and ultrapure water was poured. Then, DPV measurement of Step 3 was executed.
In the above, the same procedure was executed by changing the amount of the mixed solution containing S. aureus and labeled antibody therefor to 0.2 μL, 0.5 μL, 1.0 μL, or 2.5 μL.

2.3 (Condition C: E. coli O26 and E. coli O157)
1. 53 μL of a mixed solution containing E. coli O26 and the labeled antibody for E. coli O26 was added to 0 μL of a mixed solution containing E. coli O157 and the labeled antibody for E. coli O157, then mixed, and the mixed solution was dropped onto the working electrode of the measuring apparatus to stand for 5 minutes, and ultrapure water was poured. Then, DPV measurement of Step 3 was executed.
In the above, the same procedure was performed by changing the amount of the mixed solution containing E. coli O157 and labeled antibody for E. coli O157 to 0.5 μL, 1.0 μL, and 1.5 μL.

3. 30 μL of phosphate buffer solution (PB) was dropped onto the electrode (single electrode composed of working electrode, referencing electrode, and counter electrode), and DPV was measured.

The label was not attached to the electrode chip in advance.

(4) E. coli O26 could be distinguished from Salmonella under Condition A, S. aureus under Condition B, and E. coli O157 under Condition C according to the values of peak potential and peak current, and estimated quantitative values of the bacteria could also be calculated. The target determination unit calculates an estimated amount of the number of individuals of each target from a preset current response value per single cell or virus particle and a current peak (peak height).

FIG. 19A is the measured data of the peak potential and current values (peak height) for E. coli O26 and Salmonella. Estimates of Salmonella (cell number) can be determined according to the peak height. Since the E. coli O26 is a fixed quantity, the peak height is approximately the same, and an estimate of the E. coli O26 (the numbers of cells) could be obtained from the peak height. The same applies to Conditions B and C.

FIG. 19B is the measured data of the peak potential and current values (peak height) for E. coli O26 and S. aureus. Estimates (cell number) of S. aureus could be determined according to the peak height. FIG. 19C is the measured data of the peak potential and current values (peak height) for E. coli O26 and E. coli O157. Estimates (numbers of cells) of E. coli O157 could be determined according to the peak height.

In FIG. 19D, the estimated amounts (number of cells) of E. coli O26 and Salmonella are displayed in orders of magnitude. In FIG. 19E, the estimated amounts of E. coli O26 and S. aureus (cells) are displayed in orders of magnitude. In FIG. 19F, the estimated amounts of E. coli O26 and E. coli O157 (cells) are displayed in orders of magnitude. Measurement data measured by DPV detector is sent to a mobile terminal. A label determination application (program), which is installed in the mobile terminal, displays the measurement data (peak potential, peak height, background current) and the estimated amount on the display. The estimated amount is determined according to the peak height. The estimated amount is obtained by matching the peak height values of the measurement data with the numerical range of peak heights, which is contained in data for peak potential matching stored in a memory of the mobile terminal or that of the DPV detector.

### Example 10

It is shown that three kinds of bacteria in food were optically detected.

(1) Chicken mince was used as a food.

(2) The experimental procedure is as follows.

1. 5 mL of the sample liquid obtained from chicken mince was filtered twice with a 20-μm filter.

2. The filtrate was filtered through a 0.1 μm filter twice.

3. The 0.1-μm filter, on which bacteria is attached, was immersed in 2.5-mL sterile water to obtain a bacteria dispersion.

4. The number of bacteria (2.4×10⁶ CFU/mL) was counted with Petri films (37° C., 24 hours).

5. 1.5 mL of the bacterial dispersion was centrifuged (2000×g, 5 min), and then the supernatant was discarded and the remainder was dispersed in 90 μL of ultrapure water (4.0×10⁷ CFU/mL).

6. 62.5 μL of the solution prepared in Step “5” was added to a mixture of 0.5 μL each of the solution of E. coli O26, O157, and S. aureus (1×10⁹ CFU/mL each).

7. 86 μL of ultrapure water was further added and diluted (to total volume of 150 μL).

8. 50 μL each of AuNP/PANI-anti-O26 antibody, AgNP/PANI-anti-O157 antibody, and CuNP/PANI-anti-S. aureus antibody were added to the solution, and the mixture was stirred at room temperature for 30 minutes (total volume: 300 μL).

9. 1 μL of the solution was dropped onto a slide glass, and the glass was naturally dried, and then observation with a dark-field microscope was executed.

Note that E. coli O26, O157, S. aureus (1.7×10³ cells), chicken mince: 8.3×10³ cells (miscellaneous bacteria, O26, O157, and S. aureus are not included) in the drop spots of the slide glass.

FIG. 20A is an image observed by dark-field microscopy. The number “1” indicates E. coli O26 (blue), “2” indicates E. coli O157 (orange), “3” indicates S. aureus (white), and “4” indicates miscellaneous bacteria (no colored).

FIG. 20B is the wavelengths (Wavelength) and wavelength intensities (Intensity) of “1” E. coli O26, “2” E. coli O157, “3” S. aureus, and “4” miscellaneous bacteria in the images of FIG. 20A. Each bacteria type could be distinguished according to the difference in the wavelengths. Since the wavelength intensity of other bacteria (miscellaneous bacteria) is lower than that of the other bacteria types, it is possible to identify them with high accuracy by setting a threshold value.

### EXPLANATION OF REFERENCES

1 DPV detector
102 Data input/output unit
103 Display unit
104 Input-operation unit
105 Voltage-application control unit
106 Current-response measuring unit
107 Data memory unit
108 Target determination unit
11 Label
20 Electrode chip
21 Substrate
22 Working electrode
23 Reference electrode
24 Counter electrode
31 Slide glass
41 Optical cell
2 Image analysis device
3 Microscope
4 Display unit
5 Wavelength measuring means
6 Wavelength analysis device

1.-20. (canceled)

21. Two or more types of metal nanostructures comprising:
at least one of the two or more types of metal nanostructures having insulating properties; and
each of the two or more types of metal nanostructures that comprise different electrochemical properties or optical properties from each other and is capable of distinguishing two or more different types of targets by specifically binding to different targets and then detecting their electrochemical properties or optical properties;
wherein the electrochemical properties are current response which is the waveform of the current change when potential is changed within a predetermined range, or a peak potential which is the potential when the current peak is shown;
wherein the optical properties comprise a property selected from color, wavelength, absorption, fluorescence, scattering;
wherein the two or more types of metal nanostructures, among the two or more metal nanostructures,
(a) at least one metal nanostructure is a polymer composite comprising one noble metal selected from chemically stable gold nanoparticles, palladium nanoparticles, silver nanoparticles, and platinum nanoparticles,
(b) other metal nanostructures different from the metal nanostructures selected in the above (a), comprises at least one selected from below (i) through (vi),
(i) a polymer composite including metal nanoparticles different from the noble metal selected in the above (a),
(ii) a polymer composite including a noble metal selected, within the noble metal, from the noble metal different from the noble metal selected in the above (a),
(iii) metal nanoparticles different from the noble metal selected in the above (a),
(iv) noble metal nanoparticles different from the noble metal selected in the above (a),
(v) metal oxide nanoparticles, and
(vi) metal nanoparticles covered with a metal oxide film;
wherein the target comprises one or more types selected from Escherichia coli, Salmonella, Enterobacteriaceae, Staphylococcus aureus, norovirus, and influenza virus.

22. Two or more types of metal nanostructures comprising:
at least one of the two or more types of metal nanostructures having insulating properties; and
each of the two or more types of metal nanostructures that comprise different electrochemical properties or optical properties from each other and is capable of distinguishing two or more different types of targets by specifically binding to different targets and then detecting their electrochemical properties or optical properties;
wherein the electrochemical properties are current response which is the waveform of the current change when potential is changed within a predetermined range, or a peak potential which is the potential when the current peak is shown;
wherein the optical properties comprise a property selected from color, wavelength, absorption, fluorescence, scattering;
wherein the at least one metal nanostructure among the two or more metal nanostructures comprises polymer composite containing copper nanoparticles or one noble metal selected from chemically stable gold nanoparticles, palladium nanoparticles, silver nanoparticles, and platinum nanoparticles;
wherein the target comprises one or more types selected from Escherichia coli, Salmonella, Enterobacteriaceae, Staphylococcus aureus, norovirus, and influenza virus.

23. A label kit having two or more labeling solutions separately containing each of the two or more metal nanostructures of claim 21.

24. A label kit having two or more labeling solutions separately containing each of the two or more metal nanostructures of claim 22.

25. A detector comprising:
a target determination unit that compares data of electrochemical properties or optical properties detected by the label kit according to claim 23 with collation data including at least targeting data or labeling data to determine the type of target corresponding to the detected data.

26. A detector comprising:
a target determination unit that compares data of electrochemical properties or optical properties detected by the label kit according to claim 24 with collation data including at least targeting data or labeling data to determine the type of target corresponding to the detected data.

27. An electrode chip comprising:
two or more types of metal nanostructures according to claim 21.

28. An electrode chip comprising:
two or more types of metal nanostructures according to claim 22.

29. A detector comprising:
a target determination unit that compares data detected by the electrode chip according to claim 27 with collation data including at least targeting data or labeling data to determine the type of target corresponding to the detected data.

30. A detector comprising:
a target determination unit that compares data detected by the electrode chip according to claim 28 with collation data including at least targeting data or labeling data to determine the type of target corresponding to the detected data.

31. A method for producing two or more metal nanostructures according to claim 21, the method comprising:
a step of preparation of metal nanostructures in a composite including metal nanoparticles and polymer, in which monomers of conductive polymers are oxidized by metal ions in an aqueous solution, and by an oxidation-reduction reaction, the oxidized conductive polymer monomers reduce the metal ions,
a step of coating the metal nanostructures obtained in the step of preparation with an insulating material.

32. A method for producing two or more metal nanostructures according to claim 22, the method comprising:
a step of preparation of metal nanostructures in a composite including metal nanoparticles and polymer, in which monomers of conductive polymers are oxidized by metal ions in an aqueous solution, and by an oxidation-reduction reaction, the oxidized conductive polymer monomers reduce the metal ions,
a step of coating the metal nanostructures obtained in the step of preparation with an insulating material.
